(19) United States
(12) Patent Application Publication
(10) Pub. No.: US 2003/0170666 A1
(43) Pub. Date: Sep. 11, 2003

(54) Amplification

(75) Inventors: Peter Gill, Birmingham (GB); Javaid Hussain, Birmingham (GB); Adam Long, Birmingham (GB)

Correspondence Address: MERCHANT & GOULD PC, P.O. BOX 2903, MINNEAPOLIS, MN, 55402-0903, US

(73) Assignee: THE SECRETARY OF STATE FOR THE HOME, Birmingham (GB)

(21) Appl. No.: 10/225,938

(22) Filed: Aug. 21, 2002

(30) Foreign Application Priority Data
Aug. 21, 2001 (GB) 0120300.9
Apr. 11, 2002 (GB) 0208363.2

(51) Int. Cl. C12Q 1/68; C07H 21/04

(52) U.S. Cl. 435/6; 536/24.3

(57) ABSTRACT

Primer mixtures and methods of investigating single nucleotide polymorphisms in samples of DNA are provided having particular primer sequences. The primer mixtures and methods are aimed at achieving optimised performance in multiplexes.

This invention concerns improvements in and relating to amplification, particularly, but not exclusively, relating to primers, combinations of primers, multiplexes of primers, methods of using them and amplification methods to amplify DNA for analysis, particularly but not exclusively DNA including single nucleotide polymorphisms.

In forensic investigations, and analysis for other purposes, it is known to make use of bi-allelic markers or single nucleotide polymorphisms (SNP's). SNP's represent single base locations where variations between the sequence for one being and another can occur. A SNP may for instance be the presence of G or C, or of A or T, in the sequence of an individual, with some of the individuals having one of the options and other individuals having the other option. By considering a large number of such SNP's at different loci, a set of SNP results for an individual can be obtained which is useful for investigative purposes. The results may be compared with the results from another sample, compared with the statistical occurrence of that set of results within the population as a whole or used in other ways.

As each SNP can only vary between one of two options, a substantial number of different locations, loci, need to be investigated to achieve a set of results which is statistically significant in comparisons or other uses. Analysing a large number of loci to determine the identity of SNP's on them is highly time consuming if the loci are considered individually, and introduces significant compatibility and reliability problems if multiplexes are used to analyse a number of those loci simultaneously.

Methods aimed at improving the ease with which a large number of SNP's can be investigated simultaneously, and for achieving successful analysis where the original sample levels are very small, are set out in WO01/07640 of The Secretary of State for the Home Department.

The present invention has amongst its aims the provision of one or more primers suitable for use in SNP based investigations which are appropriate for forensic science based investigations in particular. The present invention has amongst its aims the provision of a combination of primers, and particularly multiplexes of primers, in which the primers are balanced with one another, the resulting amplification products can readily be distinguished from one another and the SNP's identified are of interest in a forensic context. The present invention has amongst its aims the provision of methods employing such primers, combinations of primers and multiplexes to achieve enhanced amplification and/or analysis. The present invention has amongst its aims the provision of improved amplification methods which allow for efficient amplification which is also highly selective using a minimal number of primers.

According to a first aspect of the invention we provided a mixture including at least one primer pair, at least one of those primer pairs being a primer pair having or including one of the following sequences:

| | | | |
|---|---|---|---|
| Pair 1 | Primer 1 | cgacgtggtggatgtgctagccagatgtttctcaagtggtcctg | SEQ ID NO 1 |
| | Primer 2 | tgacgtggctgacctgagacaaagtggtttctcaagtggtccca | SEQ ID NO 2 |
| Pair 2 | Primer 1 | cgacgtggtggatgtgctagcctctgctttaaccacaccagca | SEQ ID NO 4 |
| | Primer 2 | tgacgtggctgacctgagaccctctgctttaaccacaccagcc | SEQ ID NO 5 |
| Pair 3 | Primer 1 | cgacgtggtggatgtgctagaccaaccccacaaagcagg | SEQ ID NO 7 |
| | Primer 2 | tgacgtggctgacctgagacaccaaccccacaaagcagc | SEQ ID NO 8 |
| Pair 4 | Primer 1 | cgacgtggtggatgtgctagctctttccacctgaaageaaggg | SEQ ID NO 10 |
| | Primer 2 | tgacgtggctgacctgagacctctttccacctgaaagcaaggt | SEQ ID NO 11 |
| Pair 5 | Primer 1 | cgacgtggtggatgtgctagcgcagcccttgccatctg | SEQ ID NO 13 |
| | Primer 2 | tgacgtggctgacctgagaccgcagcccttgccatctc | SEQ ID NO 14 |
| Pair 6 | Primer 1 | cgacgtggtggatgtgctagcttagatgtcacctcttccatgcac | SEQ ID NO 16 |
| | Primer 2 | tgacgtggctgacctgagaccttagatgtcacctcttccatgcag | SEQ ID NO 17 |
| Pair 7 | Primer 1 | cgacgtggtggatgtgctaggcgggaggaaggaagggagg | SEQ ID NO 19 |
| | Primer 2 | tgacgtggctgacctgagacgcgggaggaaggaagggagc | SEQ ID NO 20 |
| Pair 8 | Primer 1 | cgacgtggtggatgtgctaggatgcctcttgcattgtgaacg | SEQ ID NO 22 |
| | Primer 2 | tgacgtggctgacctgagacgatgcctcttgcattgtgaacc | SEQ ID NO 23 |
| Pair 9 | Primer 1 | cgacgtggtggatgtgctagcattgtgtttcaaacgcgtgcc | SEQ ID NO 25 |
| | Primer 2 | tgacgtggctgacctgagaccattgtgtttcaaacgcgtgct | SEQ ID NO 26 |
| Pair 10 | Primer 1 | cgacgtggtggatgtgctagtgccactctgacactgatgcttg | SEQ ID NO 28 |
| | Primer 2 | tgacgtggctgacctgagactgccactctgacactgatgcttc | SEQ ID NO 29 |
| Pair 11 | Primer 1 | cgacgtggtggatgtgctagctgccttggctcccagcc | SEQ ID NO 31 |
| | Primer 2 | tgacgtggctgacctgagacctgccttggctcccagct | SEQ ID NO 32 |
| Pair 12 | Primer 1 | cgacgtggtggatgtgctageacaaagctgatctgagcacctattac | SEQ ID NO 34 |
| | Primer 2 | tgacgtggctgacctgagaceacaaagctgatctgagcacctattag | SEQ ID NO 35 |
| Pair 13 | Primer 1 | cgacgtggtggatgtgctaggctgtgggctctatgtatcatccaac | SEQ ID NO 37 |
| | Primer 2 | tgacgtggctgacctgagacgctgtgggctctatgtatcatccaag | SEQ ID NO 38 |
| Pair 14 | Primer 1 | cgacgtggtggatgtgctaggcccaatggctaatttccttacatg | SEQ ID NO 40 |
| | Primer 2 | tgacgtggctgacctgagacgcccaatggctaatttccttacatc | SEQ ID NO 41 |
| Pair 15 | Primer 1 | cgacgtggtggatgtgctaggggaaactgctgggtctgt | SEQ ID NO 43 |
| | Primer 2 | tgacgtggctgacctgagacgggaaaetgctgggtctgc | SEQ ID NO 44 |
| Pair 16 | Primer 1 | cgacgtggtggatgtgctagctgtgcatccactgcgcc | SEQ ID NO 46 |
| | Primer 2 | tgacgtggctgacctgagacctgtgcatccactgcgca | SEQ ID NO 47 |
| Pair 17 | Primer 1 | cgacgtggtggatgtgctagcctggagcatgagctgaccac | SEQ ID NO 49 |
| | Primer 2 | tgacgtggctgacctgagaccctggagcatgagctgaccaa | SEQ ID NO 50 |
| Pair 18 | Primer 1 | cgacgtggtggatgtgctagccatgcctcacctcctgcatt | SEQ ID NO 52 |
| | Primer 2 | tgacgtggctgacctgagacccatgcetcacctcctgcatc | SEQ ID NO 53 |
| Pair 19 | Primer 1 | cgacgtggtggatgtgctaggcatgccattgccaaattcc | SEQ ID NO 55 |
| | Primer 2 | tgacgtggctgacctgagacgcatgccattgccaaattct | SEQ ID NO 56 |
| Pair 20 | Primer 1 | cgacgtggtggatgtgctaggccaaccagacctcccagg | SEQ ID NO 58 |
| | Primer 2 | tgacgtggctgacctgagacgccaaccagacctcccagc | SEQ ID NO 59 |
| Pair 21 | Primer 1 | cgacgtggtggatgtgctaggggagacaggcccatgca | SEQ ID NO 61 |
| | Primer 2 | tgacgtggctgacctgagacgggagacaggcccatgct | SEQ ID NO 62 |
| Pair 22 | Primer 1 | cgacgtggtggatgtgctagcagaaaaggcaggaacctggaca | SEQ ID NO 64 |
| | Primer 2 | tgacgtggctgacctgagaccagaaaaggcaggaacctggact | SEQ ID NO 65 |
| Pair 23 | Primer 1 | cgacgtggtggatgtgctaggagccaagaatcgcagggaa | SEQ ID NO 67 |
| | Primer 2 | tgacgtggctgacctgagacgagccaagaatcgcagggat | SEQ ID NO 68 |
| Pair 24 | Primer 1 | cgacgtggtggatgtgctagtgtgcatgttccctggtgttca | SEQ ID NO 70 |
| | Primer 2 | tgacgtggctgacctgagactgtgcatgttccctggtgttct | SEQ ID NO 71 |
| Pair 25 | Primer 1 | cgacgtggtggatgtgctagggcatacccatgtgactaaatgttga | SEQ ID NO 73 |
| | Primer 2 | tgacgtggctgacctgagacggcatacccatgtgactaaatgttgt | SEQ ID NO 74 |
| Pair 26 | Primer 1 | cgacgtggtggatgtgctaggcaaggcecaaagcaaagaa | SEQ ID NO 76 |
| | Primer 2 | tgacgtggctgacctgagacgcaaggcccaaagcaaagat | SEQ ID NO 77 |
| Pair 27 | Primer 1 | cgacgtggtggatgtgctagaggacagtggcttctgtactgcta | SEQ ID NO 79 |
| | Primer 2 | tgacgtggctgacctgagacaggacagtggcttctgtactgctt | SEQ ID NO 80 |
| Pair 28 | Primer 1 | cgacgtggtggatgtgctagctggaagggctttgtttgccaa | SEQ ID NO 82 |
| | Primer 2 | tgacgtggctgacctgagacctggaagggctttgtttgccat | SEQ ID NO 83 |
| Pair 29 | Primer 1 | cgacgtggtggatgtgctaggggggtactggggagaccaa | SEQ ID NO 85 |
| | Primer 2 | tgacgtggctgacctgagacgggggtactggggagaccat | SEQ ID NO 86 |

or sequences at least 75% homologous thereto.

Various features, options and possibilities for the first aspect of the invention are set out elsewhere in this document, but include the following.

The mixture may be an amplifying mixture, particularly a PCR amplifying mixture.

The mixture may include at least two primer pairs, preferably includes at least three primer pairs, more preferably includes at least four primer pairs, still more preferably includes at least five primer pairs and ideally includes at least 8 primer pairs having one or both of the sequences listed or the pairs above. The mixture may have between 5 and 25 primer pairs, preferably between 8 and 20 primer pairs and still more preferably between 8 and 15 primer pairs.

Preferably both of the primers of a primer pair are provided with or include the sequences for a primer pair specified above in the statement of invention for the first aspect of the invention.

The mixture may include at least two primer pairs, preferably includes at least three primer pairs, more preferably includes at least four primer pairs, still more preferably includes at least five primer pairs and ideally includes at least 8 primer pairs, from the listed primer pairs of the first aspect of the invention. The mixture may have between 5 and 20 primer pairs, preferably between 8 and 20 primer pairs and still more preferably between 8 and 15 primer pairs from the listed primer pairs of the first aspect of the invention.

The mixture may include one or more other primer pairs.

Preferably the primer sequences corresponds to that listed for the primer in the statement of invention for the first aspect of the invention in one or more case. Preferably each primer provided according to the list in the statement of invention for the first aspect of the invention corresponds to the sequence as listed.

Preferably the mixture includes at least one primer pair, at least one of those primer pairs being a primer pair having or including one of the sequences of a primer pair selected from pair 1, pair 2, pair 3, pair 7, pair 8, pair 9, pair 10, pair 11, pair 12, pair 13, pair 14, pair 15, pair 16, pair 17, pair 18, pair 19, pair 20, pair 21, pair 22, pair 23, pair 24, pair 25, pair 26, pair 27, pair 28, pair 29 as listed above. Preferably the mixture includes at least two primer pairs, more preferably at least three primer pairs, still more preferably at least four primer pairs, even more preferably at least five primer pairs and ideally at least eight primer pairs from the primer pairs listed in this paragraph. The mixture may have between 5 and 20 such primer pairs, more preferably between 10 and 20 such primer pairs and ideally between 15 and 20 such primer pairs.

The primers of a pair preferably have a locus specific portion and a universal portion. Preferably the 340 end of the locus specific portion has an SNP pairing base. The SNP pairing base may be G or C or A or T. Preferably the locus specific portion is linked directly to the universal portion of the primer.

Preferably one or more of the listed sequences of the primers have locus specific portions and/or universal portions according to the following forms:

| | | | | | |
|---|---|---|---|---|---|
| Pair 1 | Primer 1 | cgacgtggtggatgtgctag (universal portion) | | | |
| | | | {close oversize parenthesis} | SEQ ID NO 1 | |
| | | ccagatgtttctcaagtggtcctg (locus specific portion) | | | |
| | Primer 2 | tgacgtggctgacctgagac (universal portion) | | | |
| | | | {close oversize parenthesis} | SEQ ID NO 2 | |
| | | aaagtggtttctcaagtggtccca (locus specific portion) | | | |
| Pair 2 | Primer 1 | cgacgtggtggatgtgctag (universal portion) | | | |
| | | | {close oversize parenthesis} | SEQ ID NO 4 | |
| | | cctctgctttaaccacaccagca (locus specific portion) | | | |
| | Primer 2 | tgacgtggctgacctgagac (universal portion) | | | |
| | | | {close oversize parenthesis} | SEQ ID NO 5 | |
| | | cctctgctttaaccacaccagcc (locus specific portion) | | | |
| Pair 3 | Primer 1 | cgacgtggtggatgtgctag (universal portion) | | | |
| | | | {close oversize parenthesis} | SEQ ID NO 7 | |
| | | accaaccccacaaagcagg (locus specific portion) | | | |
| | Primer 2 | tgacgtggctgacctgagac (universal portion) | | | |
| | | | {close oversize parenthesis} | SEQ ID NO 8 | |
| | | accaaccccacaaagcagc (locus specific portion) | | | |
| Pair 4 | Primer 1 | cgacgtggtggatgtgctag (universal portion) | | | |
| | | | {close oversize parenthesis} | SEQ ID NO 10 | |
| | | ctctttccacctgaaagcaaggg (locus specific portion) | | | |
| | Primer 2 | tgacgtggctgacctgagac (universal portion) | | | |
| | | | {close oversize parenthesis} | SEQ ID NO 11 | |
| | | ctctttccacctgaaagcaaggt (locus specific portion) | | | |
| Pair 5 | Primer 1 | cgacgtggtggatgtgctag (universal portion) | | | |
| | | | {close oversize parenthesis} | SEQ ID NO 13 | |
| | | cgcagcccttgccatctg (locus specific portion) | | | |
| | Primer 2 | tgacgtggctgacctgagac (universal portion) | | | |
| | | | {close oversize parenthesis} | SEQ ID NO 14 | |
| | | cgcagcccttgccatctc (locus specific portion) | | | |
| Pair 6 | Primer 1 | cgacgtggtggatgtgctag (universal portion) | | | |
| | | | {close oversize parenthesis} | SEQ ID NO 16 | |
| | | cttagatgtcacctcttccatgcac (locus specific portion) | | | |
| | Primer 2 | tgacgtggctgacctgagac (universal portion) | | | |
| | | | {close oversize parenthesis} | SEQ ID NO 17 | |
| | | cttagatgtcacctcttccatgcag (locus specific portion) | | | |
| Pair 7 | Primer 1 | cgacgtggtggatgtgctag (universal portion) | | | |
| | | | {close oversize parenthesis} | SEQ ID NO 19 | |
| | | gcgggaggaaggaagggagg (locus specific portion) | | | |
| | Primer 2 | tgacgtggctgacctgagac (universal portion) | | | |
| | | | {close oversize parenthesis} | SEQ ID NO 20 | |
| | | gcgggaggaaggaagggagc (locus specific portion) | | | |
| Pair 8 | Primer 1 | cgacgtggtggatgtgctag (universal portion) | | | |
| | | | {close oversize parenthesis} | SEQ ID NO 22 | |
| | | gatgcctcttgcattgtgaacg (locus specific portion) | | | |
| | Primer 2 | tgacgtggctgacctgagac (universal portion) | | | |
| | | | {close oversize parenthesis} | SEQ ID NO 23 | |
| | | gatgcctcttgcattgtgaacc (locus specific portion) | | | |
| Pair 9 | Primer 1 | cgacgtggtggatgtgctag (universal portion) | | | |
| | | | {close oversize parenthesis} | SEQ ID NO 25 | |
| | | cattgtgtttcaaacgcgtgcc (locus specific portion) | | | |
| | Primer 2 | tgacgtggctgacctgagac (universal portion) | | | |
| | | | {close oversize parenthesis} | SEQ ID NO 26 | |
| | | cattgtgtttcaaacgcgtgct (locus specific portion) | | | |
| Pair 10 | Primer 1 | cgacgtggtggatgtgctag (universal portion) | | | |
| | | | {close oversize parenthesis} | SEQ ID NO 28 | |
| | | tgccactctgacactgatgcttg (locus specific portion) | | | |
| | Primer 2 | tgacgtggctgacctgagac (universal portion) | | | |
| | | | {close oversize parenthesis} | SEQ ID NO 29 | |
| | | tgccactctgacactgatgcttc (locus specific portion) | | | |
| Pair 11 | Primer 1 | cgacgtggtggatgtgctag (universal portion) | | | |
| | | | {close oversize parenthesis} | SEQ ID NO 31 | |
| | | ctgccttggctcccagcc (locus specific portion) | | | |
| | Primer 2 | tgacgtggctgacctgagac (universal portion) | | | |
| | | | {close oversize parenthesis} | SEQ ID NO 32 | |
| | | ctgccttggctcccagct (locus specific portion) | | | |
| Pair 12 | Primer 1 | cgacgtggtggatgtgctag (universal portion) | | | |
| | | | {close oversize parenthesis} | SEQ ID NO 34 | |
| | | cacaaagctgatctgagcacctattac (locus specific portion) | | | |
| | Primer 2 | tgacgtggctgacctgagac (universal portion) | | | |
| | | | {close oversize parenthesis} | SEQ ID NO 35 | |
| | | cacaaagctgatctgagcacctattag (locus specific portion) | | | |
| Pair 13 | Primer 1 | cgacgtggtggatgtgctag (universal portion) | | | |
| | | | {close oversize parenthesis} | SEQ ID NO 37 | |
| | | gctgtgggctctatgtatcatccaac (locus specific portion) | | | |
| | Primer 2 | tgacgtggctgacctgagac (universal portion) | | | |
| | | | {close oversize parenthesis} | SEQ ID NO 38 | |
| | | gctgtgggctctatgtatcatccaag (locus specific portion) | | | |
| Pair 14 | Primer 1 | cgacgtggtggatgtgctag (universal portion) | | | |
| | | | {close oversize parenthesis} | SEQ ID NO 40 | |
| | | gcccaatggctaatttccttacatg (locus specific portion) | | | |
| | Primer 2 | tgacgtggctgacctgagac (universal portion) | | | |
| | | | {close oversize parenthesis} | SEQ ID NO 41 | |
| | | gcccaatggctaatttccttacatc (locus specific portion) | | | |
| Pair 15 | Primer 1 | cgacgtggtggatgtgctag (universal portion) | | | |
| | | | {close oversize parenthesis} | SEQ ID NO 43 | |
| | | gggaaactgctgggtctgt (locus specific portion) | | | |
| | Primer 2 | tgacgtggctgacctgagac (universal portion) | | | |
| | | | {close oversize parenthesis} | SEQ ID NO 44 | |
| | | gggaaactgctgggtctgc (locus specific portion) | | | |
| Pair 16 | Primer 1 | cgacgtggtggatgtgctag (universal portion) | | | |
| | | | {close oversize parenthesis} | SEQ ID NO 46 | |
| | | ctgtgcatccactgcgcc (locus specific portion) | | | |
| | Primer 2 | tgacgtggctgacctgagac (universal portion) | | | |
| | | | {close oversize parenthesis} | SEQ ID NO 47 | |
| | | ctgtgcatccactgcgca (locus specific portion) | | | |
| Pair 17 | Primer 1 | cgacgtggtggatgtgctag (universal portion) | | | |
| | | | {close oversize parenthesis} | SEQ ID NO 49 | |
| | | cctggagcatgagctgaccac (locus specific portion) | | | |
| | Primer 2 | tgacgtggctgacctgagac (universal portion) | | | |
| | | | {close oversize parenthesis} | SEQ ID NO 50 | |
| | | cctggagcatgagctgaccaa (locus specific portion) | | | |
| Pair 18 | Primer 1 | cgacgtggtggatgtgctag (universal portion) | | | |
| | | | {close oversize parenthesis} | SEQ ID NO 52 | |
| | | ccatgcctcacctcctgcatt (locus specific portion) | | | |
| | Primer 2 | tgacgtggctgacctgagac (universal portion) | | | |
| | | | {close oversize parenthesis} | SEQ ID NO 53 | |
| | | ccatgcctcacctcctgcatc (locus specific portion) | | | |
| Pair 19 | Primer 1 | cgacgtggtggatgtgctag (universal portion) | | | |
| | | | {close oversize parenthesis} | SEQ ID NO 55 | |
| | | gcatgccattgccaaattcc (locus specific portion) | | | |
| | Primer 2 | tgacgtggctgacctgagac (universal portion) | | | |
| | | | {close oversize parenthesis} | SEQ ID NO 56 | |
| | | gcatgccattgccaaattct (locus specific portion) | | | |
| Pair 20 | Primer 1 | cgacgtggtggatgtgctag (universal portion) | | | |
| | | | {close oversize parenthesis} | SEQ ID NO 58 | |
| | | gccaaccagacctcccagg (locus specific portion) | | | |
| | Primer 2 | tgacgtggctgacctgagac (universal portion) | | | |
| | | | {close oversize parenthesis} | SEQ ID NO 59 | |
| | | gccaaccagacctcccagc (locus specific portion) | | | |
| Pair 21 | Primer 1 | cgacgtggtggatgtgctag (universal portion) | | | |
| | | | {close oversize parenthesis} | SEQ ID NO 61 | |
| | | gggagacaggcccatgca (locus specific portion) | | | |
| | Primer 2 | tgacgtggctgacctgagac (universal portion) | | | |
| | | | {close oversize parenthesis} | SEQ ID NO 62 | |
| | | gggagacaggcccatgct (locus specific portion) | | | |
| Pair 22 | Primer 1 | cgacgtggtggatgtgctag (universal portion) | | | |
| | | | {close oversize parenthesis} | SEQ ID NO 64 | |
| | | cagaaaaggcaggaacctggaca (locus specific portion) | | | |
| | Primer 2 | tgacgtggctgacctgagac (universal portion) | | | |
| | | | {close oversize parenthesis} | SEQ ID NO 65 | |
| | | cagaaaaggcaggaacctggact (locus specific portion) | | | |
| Pair 23 | Primer 1 | cgacgtggtggatgtgctag (universal portion) | | | |
| | | | {close oversize parenthesis} | SEQ ID NO 67 | |
| | | gagccaagaatcgcagggaa (locus specific portion) | | | |
| | Primer 2 | tgacgtggctgacctgagac (universal portion) | | | |
| | | | {close oversize parenthesis} | SEQ ID NO 68 | |
| | | gagccaagaatcgcagggat (locus specific portion) | | | |
| Pair 24 | Primer 1 | cgacgtggtggatgtgctag (universal portion) | | | |
| | | | {close oversize parenthesis} | SEQ ID NO 70 | |
| | | tgtgcatgttccctggtgttca (locus specific portion) | | | |
| | Primer 2 | tgacgtggctgacctgagac (universal portion) | | | |
| | | | {close oversize parenthesis} | SEQ ID NO 71 | |
| | | tgtgcatgttccctggtgttct (locus specific portion) | | | |
| Pair 25 | Primer 1 | cgacgtggtggatgtgctag (universal portion) | | | |
| | | | {close oversize parenthesis} | SEQ ID NO 73 | |
| | | ggcatacccatgtgactaaatgttga (locus specific portion) | | | |
| | Primer 2 | tgacgtggctgacctgagac (universal portion) | | | |
| | | | {close oversize parenthesis} | SEQ ID NO 74 | |
| | | ggcatacccatgtgactaaatgttgt (locus specific portion) | | | |
| Pair 26 | Primer 1 | cgacgtggtggatgtgctag (universal portion) | | | |
| | | | {close oversize parenthesis} | SEQ ID NO 76 | |
| | | gcaaggcccaaagcaaagaa (locus specific portion) | | | |
| | Primer 2 | tgacgtggctgacctgagae (universal portion) | | | |
| | | | {close oversize parenthesis} | SEQ ID NO 77 | |
| | | gcaaggcccaaagcaaagat (locus specific portion) | | | |
| Pair 27 | Primer 1 | cgacgtggtggatgtgctag (universal portion) | | | |
| | | | {close oversize parenthesis} | SEQ ID NO 79 | |
| | | aggacagtggcttctgtactgcta (locus specific portion) | | | |
| | Primer 2 | tgacgtggctgacctgagac (universal portion) | | | |
| | | | {close oversize parenthesis} | SEQ ID NO 80 | |
| | | aggacagtggcttctgtactgctt (locus specific portion) | | | |
| Pair 28 | Primer 1 | cgacgtggtggatgtgctag (universal portion) | | | |
| | | | {close oversize parenthesis} | SEQ ID NO 82 | |
| | | ctggaagggctttgtttgccaa (locus specific portion) | | | |
| | Primer 2 | tgacgtggctgacctgagac (universal portion) | | | |
| | | | {close oversize parenthesis} | SEQ ID NO 83 | |
| | | ctggaagggeutgtttgecat (locus specific portion) | | | |
| Pair 29 | Primer 1 | cgacgtggtggatgtgctag (universal portion) | | | |
| | | | {close oversize parenthesis} | SEQ ID NO 85 | |
| | | gggggtactggggagaccaa (locus specific portion) | | | |
| | Primer 2 | tgacgtggctgacctgagac (universal portion) | | | |
| | | | {close oversize parenthesis} | | SEQ ID NO 86 |
| | | gggggtactggggagaccat (locus specific portion) | | | |

or sequences at least 75% homologous thereto in respect of the universal portion and/or

Preferably each of the above mentioned primers in the pairs are forward primers and are preferably accompanied by a reverse primer. Preferably the reverse primers for the respective pairs are as follows:

| | | |
|---|---|---|
| Pair 1— | ctagctggtg gctgtgctag gggaagctgg tggtaggaac tg | SEQ ID NO 3 |
| Pair 2— | ctagctggtg gctgtgctag gcaataccca tggtgtgtct ga | SEQ ID NO 6 |
| Pair 3— | ctagctggtg gctgtgctag gaccagtgac ctcgcttctg c | SEQ ID NO 9 |
| Pair 4— | ctagctggtg gctgtgctag gateagecac ccctctagcc | SEQ ID NO 12 |
| Pair 5— | ctagctggtg gctgtgctag ggctgtgaag tgagcctgg | SEQ ID NO 15 |
| Pair 6— | ctagctggtg gctgtgctag tttgaaagca cccaggaatg g | SEQ ID NO 18 |
| Pair 7— | ctagctggtg gctgtgctag agaggaagcc gcccctg | SEQ ID NO 21 |
| Pair 8— | ctagctggtg gctgtgctag caacgtcaac agcacaactc tgc | SEQ ID NO 24 |
| Pair 9— | ctagctggtg gctgtgctag ggatCagaga aagtgcagct gg | SEQ ID NO 27 |
| Pair 10— | ctagctggtg gctgtgctag aatggggaga ttggcttgga c | SEQ ID NO 30 |
| Pair 11— | ctagctggtg gctgtgctag cctgaacatc cctgaaggta tttcg | SEQ ID NO 33 |
| Pair 12— | ctagctggtg gctgtgctag tgcctttgag aactcagtgc ct | SEQ ID NO 36 |
| Pair 13— | ctagctggtg gctgtgctag gcaatggtga aaccacattg cag | SEQ ID NO 39 |
| Pair 14— | ctagctggtg gctgtgctag gaggactgta eagcagcgtc agtg | SEQ ID NO 42 |
| Pair 15— | ctagctggtg gctgtgctag cagctcttga ccctggctg | SEQ ID NO 45 |
| Pair 16— | ctagctggtg gctgtgctag ctcagtggaa gtgtggcctt g | SEQ ID NO 48 |
| Pair 17— | ctagctggtg gctgtgctag tggaagtccc ttccagcatc t | SEQ ID NO 51 |
| Pair 18— | ctagctggtg gctgtgctag caatcctgtt tgcagagttc cag | SEQ ID NO 54 |
| Pair 19— | ctagctggtg gctgtgctag tgagccaagg tgtgggga | SEQ ID NO 57 |
| Pair 20— | ctagctggtg gctgtgctag aaacacaggt ctccagcttg agc | SEQ ID NO 60 |
| Pair 21— | ctagctggtggctgtgctaggccattcagaactaactagtctggga | SEQ ID NO 63 |
| Pair 22— | ctagctggtggctgtgctagcgacgggggttgagtggttca | SEQ ID NO 66 |
| Pair 23— | ctagctggtggctgtgCtaggctaaagcagctctgaaaccca | SEQ ID NO 69 |
| Pair 24— | ctagctggtggctgtgctagaacggccttgcttcgctga | SEQ ID NO 72 |
| Pair 25— | ctagctggtggctgtgctagatggctgttagtgtgcctatcatca | SEQ ID NO 75 |
| Pair 26— | ctagctggtggctgtgctagtggatagatgatcagtctgcgttct | SEQ ID NO 78 |
| Pair 27— | ctagctggtggctgtgctagggccagcagagattcacactgt | SEQ ID NO 81 |
| Pair 28— | ctagctggtggctgtgctaggatggaatcactgtccttgccct | SEQ ID NO 84 |
| Pair 29— | ctagctggtggctgtgctagcggaggagattttgccctgca | SEQ ID NO 87 |

According to a second aspect of the invention we provide a mixture of primers pairs, the mixture including at least one primer pair which has at least one primer which hybridises to a locus, the locus being one of the following loci according to their GenBank designation:

| | Locus | Chromosome |
|---|---|---|
| 2) | AC007491.4_74646 | 16 |
| 3) | NT 001420.0_678426 | 7 |
| 4) | AC005691.1_106668 | 17 |
| 5) | Z97056.1_50018 | 22 |
| 6) | NT 000927.0_6463 | 19 |
| 7) | AC011742.3_129514 | 2 |
| 8) | AF186998.2_5696 | 8 |
| 10) | AP001721.1_327246 | 21 |
| 11) | AC006541.1_9220 | 19 |
| 12) | AC007050.25_96304 | 22 |
| 13) | AC019334.4_170838 | 13 |
| 14) | AC027107.4_130807 | 4 |
| 15) | AC066583.2_87058 | 3 |
| 16) | AC008165.3_9432 | 8 |
| 17) | AC005659.3_91393 | 10 |
| 18) | AC004228.2_133746 | 11 |
| 19) | AC010792.4_15930 | 17 |
| 20) | AC021582.5_178322 | 9 |
| 21) | AC024437.3 | 3 |
| 22) | AC068999.10_1513401 | 18 |
| 23) | AC024186.4 | 5 |
| 24) | AC022927.2 | 15 |
| 25) | AC009950.6 | 2 |
| 26) | AC008648.5 | 5 |
| 27) | AC026235.12 | 1 |
| 28) | AC023551.3 | 1 |
| 29) | AL138846.3 | 1 |

Various features, options and possibilities from the second aspect of the invention are set out elsewhere in this document, but include the following.

The mixture may be an amplifying mixture, particularly a PCR amplifying mixture.

The mixture may include at least two primer pairs, preferably includes at least three primer pairs, more preferably includes at least four primer pairs, still more preferably includes at least five primer pairs and ideally includes at least 8 primer pairs each targeting a separate locus from the list. The mixture may have between 5 and 25 primer pairs, preferably between 8 and 20 primer pairs and still more preferably between 8 and 15 primer pairs each targeting a separate locus from the list.

The mixture may include one or more other primer pairs.

Preferably one primer of a primer pair hybridises to a locus given one identity for the nucleotide of the SNP and the other primer of that primer pair hybridises to that locus given the other identity of the SNP.

Preferably the at least one primer pair is selected from loci 1 to 3, 7, 8, 10 to 29 identified in the statement of invention of the second aspect of the invention. More preferably at least two, still more preferably at least three, more preferably at least four, still more preferably at least five and ideally at least eight primer pairs from the listing of this paragraph are provided. The mixture may have between 5 and 25 primer pairs.

According to a third aspect of the invention we provide a mixture of primer pairs, wherein the mixture including one or more primer pairs which has at least one primer which hybridises to a locus, the locus being one or more of the following loci according to their SNP consortium designation:

| | Locus | Chromosome |
|---|---|---|
| 2) | TSC 0621704 (M2) | 16 |
| 3) | TSC 0846740 (Y3) | 7 |
| 4) | TSC 0098550 (K) | 17 |
| 5) | TSC 0929531 (J3) | 22 |
| 6) | TSC 0229435 (O3) | 19 |
| 7) | TSC 0255737 (K4) | 2 |
| 8) | TSC 0421768 (A4) | 8 |
| 9) | TSC 0086795 (Z2) | 6 |
| 10) | TSC 0078283 (K3) | 21 |
| 11) | TSC 0156245 (J2) | 19 |
| 12) | TSC 1088189 (I3) | 22 |
| 13) | TSC 0091355 (D4) | 13 |
| 14) | TSC 0091355 (D4) | 4 |
| 15) | TSC 0091355 (D4) | 3 |
| 16) | TSC 0252540 (D) | 8 |
| 17) | TSC 0031988 (X) | 10 |
| 18) | TSC 0155410 (F) | 11 |
| 19) | TSC 0384808 (L2) | 17 |
| 20) | TSC 0820041 (W3) | 9 |
| 21) | TSC 1342445 (B6) | 3 |
| 22) | TSC 1156239 (N4) | 18 |
| 23) | TSC 1588825 (O6) | 5 |
| 24) | TSC 0434817 (L6) | 15 |
| 25) | TSC 1773816 (Z5) | 2 |
| 26) | TSC 0746324 (U6) | 5 |
| 27) | TSC 0000254 (W5) | 1 |
| 28) | TSC 0298072 (U5) | 1 |
| 29) | TSC 0176551 (P5) | 1 |

Various features, options and possibilities from the third aspect of the invention are set out elsewhere in this document, but include the following.

The mixture may be an amplifying mixture, particularly a PCR amplifying mixture.

The mixture may include at least two primer pairs, preferably includes at least three primer pairs, more preferably includes at least four primer pairs, still more preferably includes at least five primer pairs and ideally includes at least 8 primer pairs each targeting a separate locus from the list. The mixture may have between 5 and 25 primer pairs, preferably between 8 and 20 primer pairs and still more preferably between 8 and 15 primer pairs each targeting a separate locus from the list.

The mixture may include one or more other primer pairs.

Preferably one primer of a primer pair hybridises to a locus given one identity for the nucleotide of the SNP and the other primer of that primer pair hybridises to that locus given the other identity of the SNP.

Preferably the at least one primer pair is selected from loci 1 to 3, 7 to 29 identified in the statement of invention of the second aspect of the invention. More preferably at least two, still more preferably at least three, more preferably at least four, still more preferably at least five and ideally at least eight primer pairs from the listing of this paragraph are provided. The mixture may have between 5 and 25 primer pairs.

According to a fourth aspect of the invention we provide a method of investigating single nucleotide polymorphisms in a sample of DNA, the method comprising contacting the DNA containing sample with at least one set of first primers, amplifying the DNA using those primers to give an amplified product, contacting at least a portion of the amplified product with at least one second set of primers, amplifying the DNA using those second set of primers to give a further amplified product and examining one or more characteristics of the further amplified product, in which the first primers include one or more pairs of primers pairs, at least one of those primer pairs being a primer pair having or including the following sequences:

| | | | |
|---|---|---|---|
| Pair 1 | Primer 1 | cgacgtggtggatgtgctagccagatgtttctcaagtggtcctg | SEQ ID NO 1 |
| | Primer 2 | tgacgtggctgacctgagacaaagtggtttctcaagtggtccca | SEQ ID NO 2 |
| Pair 2 | Primer 1 | cgacgtggtggatgtgctagcctctgctttaaccacaccagca | SEQ ID NO 4 |
| | Primer 2 | tgacgtggctgacctgagaccctctgctttaaccacaccagcc | SEQ ID NO 5 |
| Pair 3 | Primer 1 | cgacgtggtggatgtgctagaccaaccccacaaagcagg | SEQ ID NO 7 |
| | Primer 2 | tgacgtggctgacctgagacacoaaccccacaaagcagc | SEQ ID NO 8 |
| Pair 4 | Primer 1 | cgacgtggtggatgtgctagctctttccacctgaaagcaaggg | SEQ ID NO 10 |
| | Primer 2 | tgacgtggctgacctgagacctctttccacctgaaagcaaggt | SEQ ID NO 11 |
| Pair 5 | Primer 1 | cgacgtggtggatgtgctagcgcagcccttgccatctg | SEQ ID NO 13 |
| | Primer 2 | tgacgtggctgacctgagaccgcagcccttgccatctc | SEQ ID NO 14 |
| Pair 6 | Primer 1 | cgacgtggtggatgtgctagcttagatgtcacctcttccatgcac | SEQ ID NO 16 |
| | Primer 2 | tgacgtggctgacctgagaccttagatgtcacctcttccatgcag | SEQ ID NO 17 |
| Pair 7 | Primer 1 | cgacgtggtggatgtgctaggcgggaggaaggaagggagg | SEQ ID NO 19 |
| | Primer 2 | tgacgtggctgacctgagacgcgggaggaaggaagggagc | SEQ ID NO 20 |
| Pair 8 | Primer 1 | cgacgtggtggatgtgctaggatgcctcttgcattgtgaacg | SEQ ID NO 22 |
| | Primer 2 | tgacgtggctgacctgagacgatgcctcttgcattgtgaacc | SEQ ID NO 23 |
| Pair 9 | Primer 1 | cgacgtggtggatgtgctagcattgtgtttcaaacgcgtgcc | SEQ ID NO 25 |
| | Primer 2 | tgacgtggctgacctgagaccattgtgtttcaaacgcgtgct | SEQ ID NO 26 |
| Pair 10 | Primer 1 | cgacgtggtggatgtgctagtgccactctgacactgatgcttg | SEQ ID NO 28 |
| | Primer 2 | tgacgtggctgacctgagactgccactctgacactgatgcttc | SEQ ID NO 29 |
| Pair 11 | Primer 1 | cgacgtggtggatgtgctagctgccttggctcccagcc | SEQ ID NO 31 |
| | Primer 2 | tgacgtggctgacetgagacctgcottggctcccagct | SEQ ID NO 32 |
| Pair 12 | Primer 1 | cgacgtggtggatgtgctagcacaaagctgatctgagcacctattac | SEQ ID NO 34 |
| | Primer 2 | tgacgtggctgacctgagaccacaaagctgatctgagcacctattag | SEQ ID NO 35 |
| Pair 13 | Primer 1 | cgacgtggtggatgtgctaggctgtgggctctatgtatcatccaac | SEQ ID NO 37 |
| | Primer 2 | tgacgtggctgacctgagacgctgtgggctctatgtatcatccaag | SEQ ID NO 38 |
| Pair 14 | Primer 1 | cgacgtggtggatgtgctaggcccaatggctaatttccttacatg | SEQ ID NO 40 |
| | Primer 2 | tgacgtggctgacctgagacgcccaatggctaatttccttacatc | SEQ ID NO 41 |
| Pair 15 | Primer 1 | cgacgtggtggatgtgctaggggaaactgctgggtctgt | SEQ ID NO 43 |
| | Primer 2 | tgacgtggctgacctgagacgggaaactgctgggtctgc | SEQ ID NO 44 |
| Pair 16 | Primer 1 | cgacgtggtggatgtgctagctgtgcatccactgcgcc | SEQ ID NO 46 |
| | Primer 2 | tgacgtggctgacctgagacctgtgcatccactgcgca | SEQ ID NO 47 |
| Pair 17 | Primer 1 | cgacgtggtggatgtgctagcctggagcatgagctgaccac | SEQ ID NO 49 |
| | Primer 2 | tgacgtggctgacctgagaecctggageatgagctgaccaa | SEQ ID NO 50 |
| Pair 18 | Primer 1 | cgacgtggtggatgtgctagceatgcctcacctcctgcatt | SEQ ID NO 52 |
| | Primer 2 | tgacgtggctgacctgagacccatgcctcacctcctgcatc | SEQ ID NO 53 |
| Pair 19 | Primer 1 | cgacgtggtggatgtgctaggcatgccattgccaaattcc | SEQ ID NO 55 |
| | Primer 2 | tgacgtggctgacctgagacgcatgccattgccaaattct | SEQ ID NO 56 |
| Pair 20 | Primer 1 | cgacgtggtggatgtgctaggccaaccagacctcccagg | SEQ ID NO 58 |
| | Primer 2 | tgacgtggctgacctgagacgccaaceagacctcccagc | SEQ ID NO 59 |
| Pair 21 | Primer 1 | cgacgtggtggatgtgctaggggagacaggcccatgca | SEQ ID NO 61 |
| | Primer 2 | tgacgtggctgacctgagacgggagacaggcccatgct | SEQ ID NO 62 |
| Pair 22 | Primer 1 | cgacgtggtggatgtgctagcagaaaaggeaggaacctggaca | SEQ ID NO 64 |
| | Primer 2 | tgacgtggctgacctgagaccagaaaaggcaggaacctggact | SEQ ID NO 65 |
| Pair 23 | Primer 1 | cgacgtggtggatgtgctaggagccaagaatcgcagggaa | SEQ ID NO 67 |
| | Primer 2 | tgacgtggctgacctgagacgagccaagaatcgcagggat | SEQ ID NO 68 |
| Pair 24 | Primer 1 | cgacgtggtggatgtgctagtgtgcatgttccctggtgttca | SEQ ID NO 70 |
| | Primer 2 | tgacgtggctgacctgagactgtgcatgttccctggtgttct | SEQ ID NO 71 |
| Pair 25 | Primer 1 | cgacgtggtggatgtgctagggcatacccatgtgactaaatgttga | SEQ ID NO 73 |
| | Primer 2 | tgacgtggctgacutgagaeggcatacccatgtgactaaatgttgt | SEQ ID NO 74 |
| Pair 26 | Primer 1 | cgacgtggtggatgtgctaggcaaggcccaaagcaaagaa | SEQ ID NO 76 |
| | Primer 2 | tgacgtggctgacctgagacgcaaggcccaaagcaaagat | SEQ ID NO 77 |
| Pair 27 | Primer 1 | cgacgtggtggatgtgctagaggacagtggcttctgtactgcta | SEQ ID NO 79 |
| | Primer 2 | tgacgtggctgacctgagacaggacagtggcttctgtactgctt | SEQ ID NO 80 |
| Pair 28 | Primer 1 | cgacgtggtggatgtgctagctggaagggctttgtttgccaa | SEQ ID NO 82 |
| | Primer 2 | tgacgtggctgacctgagacctggaagggctttgtttgccat | SEQ ID NO 83 |
| Pair 29 | Primer 1 | cgacgtggtggatgtgctaggggggtactggggagaccaa | SEQ ID NO 85 |
| | Primer 2 | tgacgtggctgacctgagacgggggtactggggagaccat | SEQ ID NO 86 |

or sequences 75% homologous therewith.

Preferably the mixture includes at least one primer pair, at least one of those primer pairs being a primer pair having or including one of the sequences of a primer pair selected from pair 1, pair 2, pair 3, pair 7, pair 8, pair 9, pair 10, pair 11, pair 12, pair 13, pair 14, pair 15, pair 16, pair 17, pair 18, pair 19, pair 20, pair 21, pair 22, pair 23, pair 24, pair 25, pair 26, pair 27, pair 28, pair 29 as listed above. Preferably the mixture includes at least two primer pairs, more preferably at least three primer pairs, still more preferably at least four primer pairs, even more preferably at least five primer pairs and ideally at least eight primer pairs from the primer pairs listed in this paragraph. The mixture may have between 5 and 20 such primer pairs, more preferably between 10 and 20 such primer pairs and ideally between 15 and 20 such primer pairs.

Various other features, options or possibilities for the fourth aspect of the invention are set out elsewhere in this document.

According to a fifth aspect of the invention we provide a method of investigating single nucleotide polymorphisms in a sample of DNA, the method comprising contacting the DNA containing sample with at least one set of first primers, amplifying the DNA using those primers to give an amplified product, contacting at least a portion of the amplified product with at least one second set of primers, amplifying the DNA using those second set of primers to give a further amplified product and examining one or more characteristics of the further amplified product, in which the first primers include one or more pairs of primers which have a primer which hybridises with a loci selected from the following list, the loci being identified according to their GenBank designation:

| | |
|---|---|
| 2) | AC007491.4_74646; |
| 3) | NT 001420.0_678426; |
| 4) | AC005691.1_106668; |
| 5) | Z97056.1_50018; |
| 6) | NT 000927.0_6463; |
| 7) | AC011742.3_129514; |
| 8) | AF186998.2_5696; |
| 10) | AP001721.1_327246; |
| 11) | AC006541.1_9220; |
| 12) | AC007050.25_96304; |
| 13) | AC019334.4_170838; |
| 14) | AC027107.4_130807; |
| 15) | AC066583.2_87058; |
| 16) | AC008165.3_9432; |
| 17) | AC005659.3_91393; |
| 18) | AC004228.2_133746; |
| 19) | AC010792.4_15930; |
| 20) | AC021582.5_178322. |
| 21) | AC024437.3 |
| 22) | AC068999.10_1513401 |
| 23) | AC024186.4 |
| 24) | AC022927.2 |
| 25) | AC009950.6 |
| 26) | AC008648.5 |
| 27) | AC026235.12 |
| 28) | AC023551.3 |
| 29) | AL138846.3 |

Preferably the at least one primer pair is selected from loci 1 to 3, 7, 8, 10 to 29 identified in the statement of invention of the second aspect of the invention. More preferably at least two, still more preferably at least three, more preferably at least four, still more preferably at least five and ideally at least eight primer pairs from the listing of this paragraph are provided. The mixture may have between 5 and 25 primer pairs.

Various other features, options or possibilities for the fifth aspect of the invention are set out elsewhere in this document.

According to a sixth aspect of the invention we provide a method of investigating single nucleotide polymorphisms in a sample of DNA, the method comprising contacting the DNA containing sample with at least one set of first primers, amplifying the DNA using those primers to give an amplified product, contacting at least a portion of the amplified product with at least one second set of primers, amplifying the DNA using those second set of primers to give a further amplified product and examining one or more characteristics of the further amplified product, in which the first primers include one or more pairs of primers including a primer which hybridises to one or more of the following loci, identified according to their SNP Consortium designation:

| | |
|---|---|
| 2) | TSC 0621704 (M2) |
| 3) | TSC 0846740 (Y3) |
| 4) | TSC 0098550 (K) |
| 5) | TSC 0929531 (J3) |
| 6) | TSC 0229435 (O3) |
| 7) | TSC 0255737 (K4) |
| 8) | TSC 0421768 (A4) |
| 9) | TSC 0086795 (Z2) |
| 10) | TSC 0078283 (K3) |
| 11) | TSC 0156245 (J2) |
| 12) | TSC 1088189 (I3) |
| 13) | TSC 0091355 (D4) |
| 14) | TSC 0091355 (D4) |
| 15) | TSC 0091355 (D4) |
| 16) | TSC 0252540 (D) |
| 17) | TSC 0031988 (X) |
| 18) | TSC 0155410 (F) |
| 19) | TSC 0384808 (L2) |
| 20) | TSC 0820041 (W3) |
| 21) | TSC 1342445 (B6) |
| 22) | TSC 1156239 (N4) |
| 23) | TSC 1588825 (O6) |
| 24) | TSC 0434817 (L6) |
| 25) | TSC 1773816 (Z5) |
| 26) | TSC 0746324 (U6) |
| 27) | TSC 0000254 (W5) |
| 28) | TSC 0298072 (U5) |
| 29) | TSC 0176551 (P5) |

Preferably the at least one primer pair is selected from loci 1 to 3, 7 to 29 identified in the statement of invention of the second aspect of the invention. More preferably at least two, still more preferably at least three, more preferably at least four, still more preferably at least five and ideally at least eight primer pairs from the listing of this paragraph are provided. The mixture may have between 5 and 25 primer pairs.

Various other features, options or possibilities for the sixth aspect of the invention are set out elsewhere in this document.

According to a seventh aspect of the invention we provide a method of investigating single nucleotide polymorphisms in a sample of DNA, the method comprising contacting the DNA containing sample with at least one first set of primers, a amplifying the DNA using those primers to give an amplified product, contacting at least a portion of the amplified product with at least one second set of primers, amplifying the DNA using those second set of primers to give a further amplified product and examining one or more characteristics of the further amplified product, one or more of the first sets of primers including a primer having a locus-specific portion and a further portion, one or more of the second sets of primers having a primer including a second further portion, wherein the sequence of the further portion and the second further portion corresponds.

Preferably the universal portion sequence of each forward primer in a first set of primers, ideally both forward primers, are of different sequence to one another. Preferably the universal portions have a length within one or two bases of one another and most preferably are of the same length. Preferably at least 70% of the bases in one primer's universal portion are different from the corresponding position bases in another primer's universal portion of the first set. Preferably the sequences are entirely different from one another. Preferably the universal portion of one forward primer will not hybridise with the same sequence as the universal portion of another forward primer of the first set, ideally the other forward primer of the first set.

Preferably at least one, ideally only one, forward primer of two or more of the first sets of primers are provided with universal portions which correspond to one another. Preferably the universal portions correspond in the sense of hybridising to the same sequence as one another. Preferably the universal portions correspond in the sense of being at least 80% homologous with one another in sequence. More preferably the universal portions correspond in the sense of being identical in sequence to one another. Preferably each of the first sets of primers is provided with at least one forward primer which has a universal portion of corresponding sequence to a primer in the other sets. Preferably a second forward primer in one or more of the first sets of primers, ideally all sets, is provided with a universal portion which corresponds to the universal portion of one or more other second primers of the first sets. Preferably each of these first sets of primers includes a primer having a first universal portion sequence and a primer having a second universal portion sequence. Preferably at least two such first sets, more preferably at least three such first sets, still more preferably at least four such first sets and potentially even five or more such first sets may be provided. Between five and twenty such first sets may each be provided with a first primer having a first universal portion sequence and a second primer having a second universal portion sequence, the first and second sequences being different.

It is particularly preferred that a first set of primers is provided for each locus under investigation, that each of the first sets of primers includes two forward primers and a reverse primer, and that the further portions of the first primer of each of the first sets of primers has a further portion of corresponding sequence and that the second forward primer of each of the first sets of primers has a further portion of corresponding sequence. It is particularly preferred also that the second set of primers includes a first and second forward primer and a reverse primer. Most preferably the first primer of the second set has a second further portion which corresponds to the further portion of the first primer of each of the first sets of primers. Most preferably the second primer of the second set has a second further portion which corresponds to the further portion of the second primers of each of the first sets of primers. Ideally only one second set of primers is provided.

Various other features, options or possibilities for the seventh aspect of the invention are set out elsewhere in this document.

The first and/or second and/or third and/or fourth and/or fifth and/or sixth and/or seventh aspects of the invention may further provide or include the following features, options or possibilities.

The percentage of homology preferable refers to the percentage of bases which are the same and at the same position. Preferably the sequences are at least 85% homologous, more preferably at least 90% homologous and ideally at least 95% homologous.

Preferably the SNP consortium designation is the SNP consortium designation on Aug. 20, 2001.

In one embodiment of the invention one or more, preferably all, of the first sets of primers may include two forward primers and a reverse primer. One or more, preferably all, of the first sets of primers may consist of two forward primers and a reverse primer. The forward primers and reverse primer preferably include sequences which anneal to the 340 and 540 sides respectively of the SNP at the locus incorporating the SNP under investigation.

The first set of primers may include one or more primers including a locus specific portion and a universal portion. Preferably the forward primers are so provided. Preferably the universal portion is attached to the 540 end of the locus specific portion, particularly in the case of forward primers. The 340 end of the forward primer is preferably provided with a SNP identifying portion. The universal portion is preferably attached directly to the locus specific portion.

In one embodiment of the invention the locus specific portion preferably includes a sequence which matches the sequence of the locus sequence in the vicinity of the SNP under investigation. More preferably the sequence matches the locus sequence for the locus sequence adjacent to the SNP under investigation, ideally up to and including the nucleotide before the SNP on the 340 side of the SNP. Preferably the forward primers of a pair of the first set of primers are provided with identical sequences for the locus specific portion. Preferably they have different SNP identifying portions.

The universal portion preferably includes a sequence which does not match the locus sequence on the locus's 340 side of the locus sequence matching the locus specific portion of the primer. More preferably the sequence does not match the sequence of the locus in the vicinity of the SNP under investigation. Ideally the sequence does not anneal to, and particularly does not match, the sequence of any published part, ideally any part, of the entire DNA sequence of the entity from which the DNA containing the SNP under investigation was obtained, for instance Homo Sapiens. The inability of the sequence of the universal portion to amplify human DNA is a particularly preferred feature.

Preferably the universal portion sequence of each forward primer in a first set of primers, ideally both forward primers, are of different sequence to one another. Preferably the universal portions have a length within one or two bases of one another and most preferably are of the same length. Preferably at least 70% of the bases in one primer's universal portion are different from the corresponding position bases in another primer's universal portion of the first set. Preferably the sequences are entirely different from one another. Preferably the universal portion of one forward primer will not hybridise with the same sequence as the universal portion of another forward primer of the first set, ideally the other forward primer of the first set.

Preferably at least one, ideally only one, forward primer of two or more of the first sets of primers are provided with universal portions which correspond to one another. Preferably the universal portions correspond in the sense of hybridising to the same sequence as one another. More preferably the universal portions correspond in the sense of being identical in sequence to one another. Preferably each of the first sets of primers is provided with at least one forward primer which has a universal portion of corresponding sequence to a primer in the other sets. Preferably a second forward primer in one or more of the first sets of primers, ideally all sets, is provided with a universal portion which corresponds to the universal portion of one or more other second primers of the first sets. Preferably each of these first sets of primers includes a primer having a first universal portion sequence and a primer having a second universal portion sequence. Preferably at least two such first sets, more preferably at least three such first sets, still more preferably at least four such first sets and potentially even five or more such first sets may be provided. Between five and twenty such first sets may each be provided with a first primer having a first universal portion sequence and a second primer having a second universal portion sequence, the first and second sequences being different.

Preferably the universal portion forms the 540 end of the forward primers of the first set.

Preferably during amplification the SNP related portion results in the amplified copies of the locus incorporating the SNP having an SNP repeat introduced into them. Ideally, the repeat has a base identity identical to that of the SNP.

Preferably the locus specific portion and SNP identifying portion of one of the forward primers anneals to the 340 side of the locus having the SNP under investigation. Preferably the locus specific portion and SNP identifying portion of another, ideally the other, of the forward primers does not anneal to the 340 side of the SNP under investigation. Preferably the annealing primer anneals due to a match between the SNP identifying portion and the SNP site, (for instance C matching to G). Preferably the non-annealing primer does not anneal due to a mis-match between the SNP identifying portion and the SNP site, (for instance, T mis-matching with T).

The SNP under investigation may be a location with variation between individuals of any two bases selected from C or G or A or T nucleotides. For instance, the SNP under investigation may be a location with variation between individuals of either a T or A nucleotide, T or C nucleotide, T or G nucleotide, A or C nucleotide, A or G nucleotide or C or G nucleotide. One possible variation may be investigated at one or more sites, with one or more other potential variations being investigated at one or more other sites.

The sample may be a sample of DNA extracted from a collected source.

The sample may be contacted with the first primer set by mixing the sample and primers together.

The sample may be a mixture. One or more contributions to the sample may be analysed as the sample itself using the present invention. The mixed sample may include male and female DNA. One of the sexes of DNA, particularly the male, may be present in low concentrations relative to the other sex. For instance, the minor sex DNA contribution may form less than 1% of the sample, potentially less than 0.1% and even less than 0.05%. The sample may contain samples from two or more sources. The method may investigate the minor sample in a mixture from two or more sources. The minor sample may form less than 1% of the mixed sample, potentially less than 0.1% of the mixed sample and even less than 0.05% of the mixed sample.

The investigation may indicate the amount of DNA in a mixed sample from one or more of the sources. The indication may be based on a comparison of the experimentally determined results, for instance the level of a distinctive unit present, compared with a set of calibration results based on investigation of known amounts of DNA in a sample.

The first amplification is preferably performed by PCR. The amplification preferably involves between 18 to 60 cycles, more preferably 20 to 40 cycles.

The amplification cycles, particularly where the first and second amplification processes are used, may have the following characteristics. Preferably the amplification cycles include a first cycle set in which the annealing temperature of the cycle is similar or above the melting temperature of the first set of primers, particularly of the locus specific portion of the first set of primers and/or similar or above the second set of primer. The amplification cycles may include a second set of cycles, with preferably, the annealing temperature in the second set of cycles being similar or below the melting temperature of the first set of primers and/or above the melting temperature of the second set of primers. The melting temperature of the first set of primers may rise after one or two cycles. The amplification cycles may include a third set of cycles, with, preferably, the annealing temperature in the third set of cycles being below the melting temperature of the first set of primers and/or similar or above the melting temperature of the second set of primers.

It is preferred that the first set of cycles provide between 2 and 10 cycles. It is preferred that the second set of cycles provide between 3 and 15 cycles. It is preferred that the third set of cycles provide between 15 and 35 cycles. Preferably the total of cycles provided in the first, second and third sets does not exceed 40 cycles.

It is preferred that the denaturation temperature for the first and/or second and/or third set of cycles be 92 to 96° C., ideally 94° C.

It is preferred that the annealing temperature for the first and/or second and/or third set of cycles be between 60 and 62 °C., ideally 61 °C. It is preferred that the annealing temperature for the second set of cycles be between 70 and 78 °C., ideally between 72 and 75 °C.

It is preferred that the extension temperature for the first and/or second and/or third set of cycles be between 70 and 75 °C., ideally 72 °C.

Amplification preferably results in extension of the annealed forward primer from its 340 end towards the 540 end of the target sequence. Amplification preferably results in extension of the reverse primer from its 340 end towards the 540 end of its target sequence. Preferably further cycles of amplification result in extension of the forward primer sequence towards the 540 end of its target, including the reverse primer sequence. Preferably further cycles of amplification result in extension of the reverse primer sequence towards the 540 end of its target, including one or more or all of the forward primer sequence and particularly the SNP identifying portion, locus specific portion, SNP related portion and further portion.

A portion of the amplified product may be removed and contacted separately with the second set of primers. Contact with the second set of primers may occur in a separate vessel to the contact with the first set of primers. This is particularly preferred where universal primers incorporating molecular beacons are used. Preferably a two tube and/or branched PCR process is used where universal primers incorporating molecular beacons are employed.

The first and second amplifications may occur in the same vessel. The first and second amplifications may occur substantially simultaneously. Preferably the method includes adding one or more of the first set of primers and one or more of the second set of primers to the sample to be amplified prior to conducting amplification cycles.

The one or more first sets of primers may be provided at a concentration of between 20 and 80 nM, more preferably between 40 and 60 nM and ideally at 50 nM+/−5%. Preferably the primers which do not compete and/or for which site overlap does not occur are provided at these levels. Where primer competition could occur and/or where primer site overlap occurs preferably the primer's relative concentrations are balanced. The reverse primer concentration for such a simultaneous process may be between 75 nM and 125 nM, for instance 100 nM+/−10%.

The second set of primers may be provided at a concentration of between 20 and 80 nM, more preferably between 40 and 60 nM and ideally at 50 nM+/−5%. The amount of the second set of primers added may be defined by Cn×L, where Cn is the concentration of the primers and L is the number of loci under consideration +/−2 and ideally is the number of loci under consideration, particularly where L is less than 100 or even less than 50. Preferably the maximum second set of primers concentration is 1000 nM.

Particularly where the first and second sets of primers are present together, it is preferred to provide the second set of primers and first set of primers at a concentration ratio of at least 5:1. A ratio of at least 10:1, more preferably at least 20:1 and ideally at least 30:1, second set concentration: first set concentration may be provided. The first set may be provided at a concentration of between 5 and 400 nM, more preferably between 10 and 200 nM. The second set may be provided at a concentration of between 300 nM and 5000 nM, more preferably between 400 and 4000 nM.

Particularly where the first and second sets of primers are present together, it is preferred to use an annealing temperature at which at least 80% of the second set of primers remain single stranded, more preferably a temperature at which at least 95% of the second set of primers remain single stranded and ideally a temperature at which at least 99% of the second set of primers remain single stranded, for some of the cycles of the amplification process. A lower annealing temperature may be used for other cycles of the amplification process. Preferably the higher temperature annealing is used at least in cycles 3 to 30, more preferably in cycles 3 to 40. A lower annealing temperature may be used in the first two cycles. A lower annealing temperature is preferably used in at least the last two cycles. The lower annealing temperature is preferably a temperature at which at least 80%, more preferably at least 90% and ideally at least 99% of the second set of primers anneal.

The amplified product may be contacted with the second primer set by mixing the sample and primers together.

The second set of primers may include one, two, three or four forward primers but ideally only include two. A reverse primer may be present, but the second set of primers may lack a reverse primer.

The invention may only provide one second set of primers, preferably including one two forward primers, and ideally a reverse primer.

One of the forward primers of the second set preferably includes a sequence which anneals to the SNP incorporating strand on the 340 side of the SNP. The reverse primer of the second set preferably includes a sequence which anneals to the 340 side of the base pairing to the SNP. Preferably the other forward primer or primers do not anneal.

In the one embodiment of the invention the second set of primers may include one or more primers including a second further portion. Preferably both the forward primers are so provided. The 540 end of the forward primer is preferably provided with a distinctive unit.

In the one embodiment of the invention the second further portion preferably includes a sequence which pairs to the sequence of the amplified product in the vicinity of the SNP identifying portion. More preferably the second further portion sequence adjacent to the SNP ideally up to and including the nucleotide before the SNP, matches the sequence of the amplified product adjacent to the SNP ideally up to and including the nucleotide before the SNP.

Preferably the sequence of the second further portion or portions does not anneal to, and particularly does not match, the sequence of any published part, ideally any part, of the entire DNA sequence of the entity from which the DNA containing the SNP under investigation was obtained, for instance Homo Sapiens. The inability of the sequence of the second further portion or portions to amplify human DNA is a particularly preferred feature.

One or more primers of a second set of primers may include a second further portion. Preferably each of the primers of a second set of primers includes a second further portion. Preferably all the second sets of primers include one or more primers including a second further portion and preferably all of each set of primers include such a second further portion. One or more of the primers may consist of the second further portion.

The second further portion of one primer of a second set of primers may differ from the second further portion of one or more of the other primers of that set of primers. Preferably each primer in a second set of primers has a different second further portion. Ideally both the forward primers of a second set of primers are provided with a different second further portion from one another. Ideally only one second set of primers, providing two forward primers and one reverse primer is provided.

The second further portion of one primer of the second set of primers may correspond to the universal portion of one or more primers in one or more of the first sets of primers. Preferably the second further portion of a primer of the second set of primers corresponds to the universal portion of a primer in each of the first sets of primers. Preferably the second set of primers includes primers which have second further portions corresponding to each of the sequences of the universal portions of each of the primers in the first set or sets of primers. Preferably the second set of primers includes two forward primers each having a second further portion which is different from one another. Preferably the second further portion of one of these primers corresponds to the universal portion provided for one of the primers in each of the first set of primers used. Preferably the other forward primer has a second further portion which corresponds to the universal portion of the other primer in each of the first sets of primers.

The 540 end of the forward primer may be provided with a distinctive unit. Preferably each forward primer of the second set of primers is provided with a different distinctive unit.

The second amplification is preferably performed by PCR. The amplification preferably involves between 18 and 30 cycles, more preferably 20 to 25 cycles.

One or more of the primers of the first and/or second set may be provided with one or more portions which are complimentary to one or more portions on one or more of the other primers in that set. The complimentary portion or portions are preferably provided in the further portion of the primers of the first set. The complimentary portion or portions are preferably provided in the second further portion of the primers of the second set. Preferably a complimentary portion is provided on each of the primers of a set. Preferably at least two complimentary portions are provided on each of the primers of a set. Preferably a complimentary portion is provided at the 340 end of a primer, ideally all the primers. Preferably a complimentary portion is provided at the 540 end of a primer, ideally all of the primers. Preferably the 340 end complimentary portion of one primer is complimentary to the 540 end complimentary portion of another primer, ideally all the other primers of the set and/or both sets. Preferably the 540 end complimentary portion of one primer is complimentary to the 340 end complimentary portion of another primer, ideally all the other primers of the set and/or both sets. A locus specific portion may be provided on the further portion including the complimentary portion or portions, particularly on the 340 end. The further portion and/or second further portion may include a sequence matching the sequence of the locus under consideration, particularly provided between two complimentary portions. The complimentary portions may be at least 3 nucleotides long, more preferably between 3 and 20 nucleotides long. The complimentary portions are preferably both of the same length. The complimentary portions may form between 5 and 40% of the further portion and/or second further portion. One, two, three or four primers of a set may be provided in this way. Preferably the reverse primer or primers are similarly provided.

The further amplified product, or a portion thereof, may be removed from the vessel in which the amplification is performed to examine the one or more characteristics. Alternatively or additionally, the one or more characteristics may be examined with the further amplified product in the vessel in which amplification is performed.

The one or more characteristic of the further amplified product may be examined by means of the presence and/or absence of a distinctive unit in the further amplified product. The distinctive unit may be incorporated in the further amplified product or be associated there with. The distinctive unit may be introduced during the amplification process and/or in a subsequent step. The subsequent step may comprise hybridisation, for instance, of a component to the SNP base. The component may be a dideoxynucleotide, particularly a dideoxynucleotide incorporating a distinctive unit such as a dye.

The distinctive unit may be a dye label or colour producing molecule.

The distinctive unit may be a sequence of DNA, for instance a molecular beacon.

Various embodiments of the invention will now be described, by way of example only, and with reference to the accompanying drawings in which:

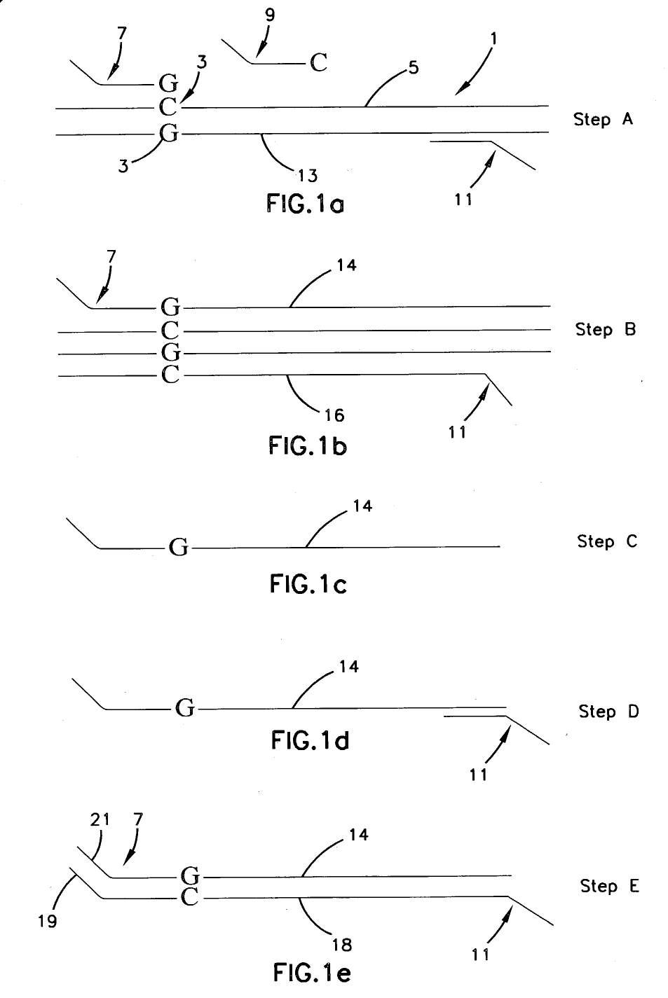

FIGS. 1a to 1e illustrates the various parts of the first stage of a process according to the present invention;

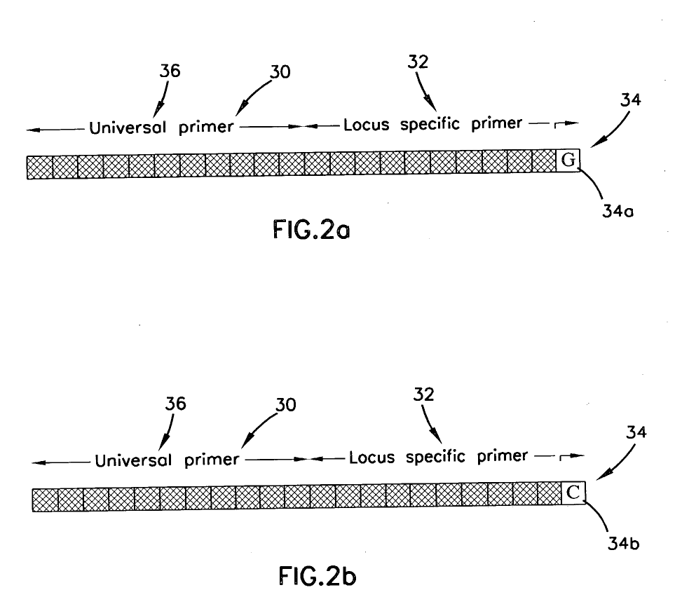

FIG. 2a illustrates one forward primer suitable for use in the present invention;

FIG. 2b illustrates a second forward primer suitable for use in the present invention and intended for use with the primer of FIG. 2a;

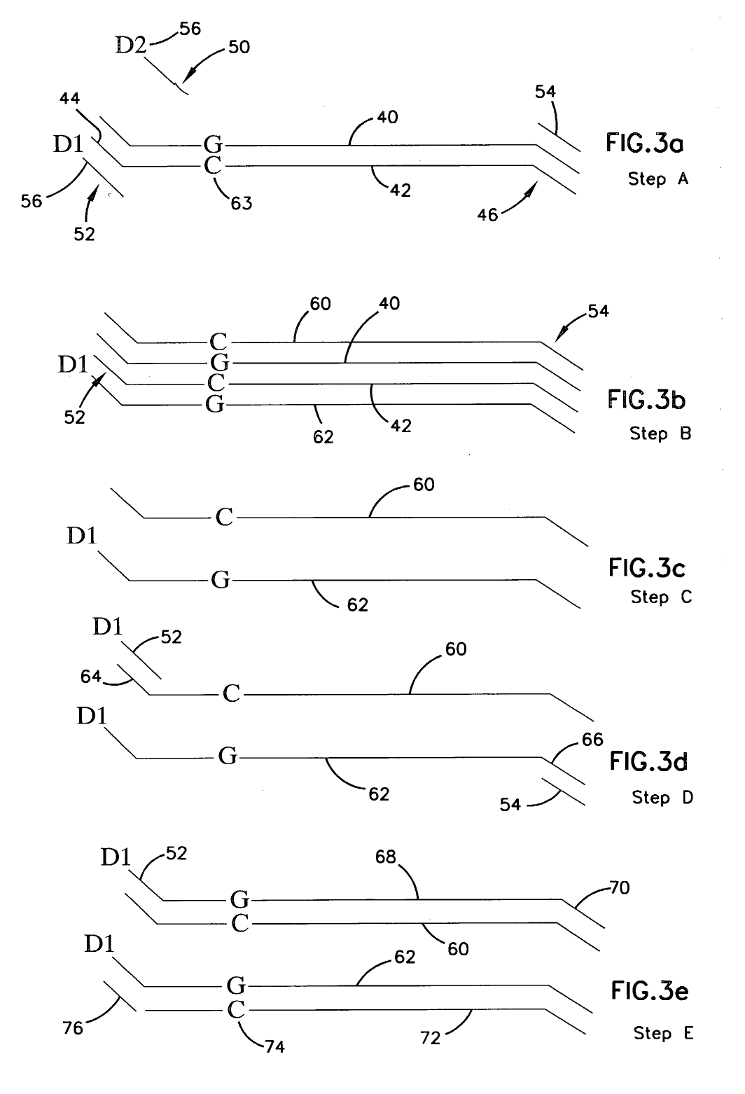

FIGS. 3a to 3e illustrates the various parts of the second stage of a process according to the present invention;

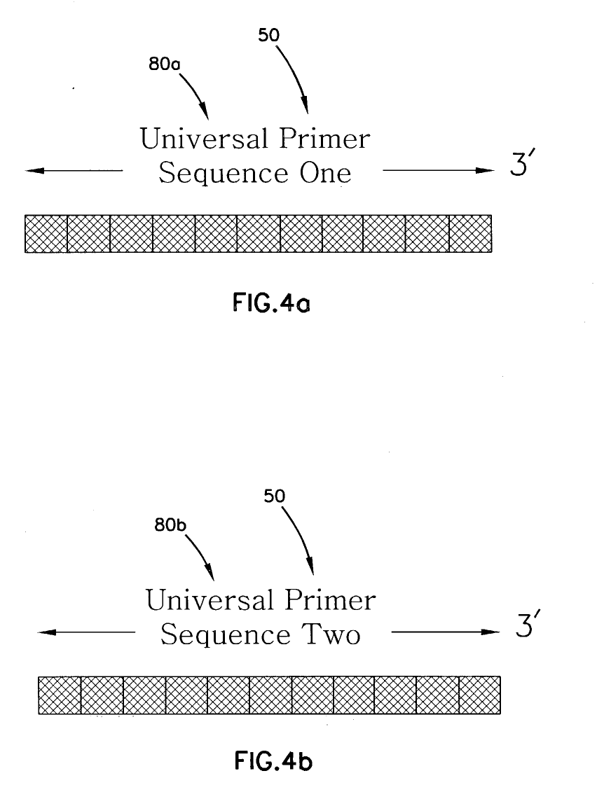

FIG. 4a illustrates a “universal” forward primer suitable for use in the second stage of the present invention;

FIG. 4b illustrates a second “universal” forward primer for use in the second stage of the present invention and intended for use with the primer of FIG. 4a;

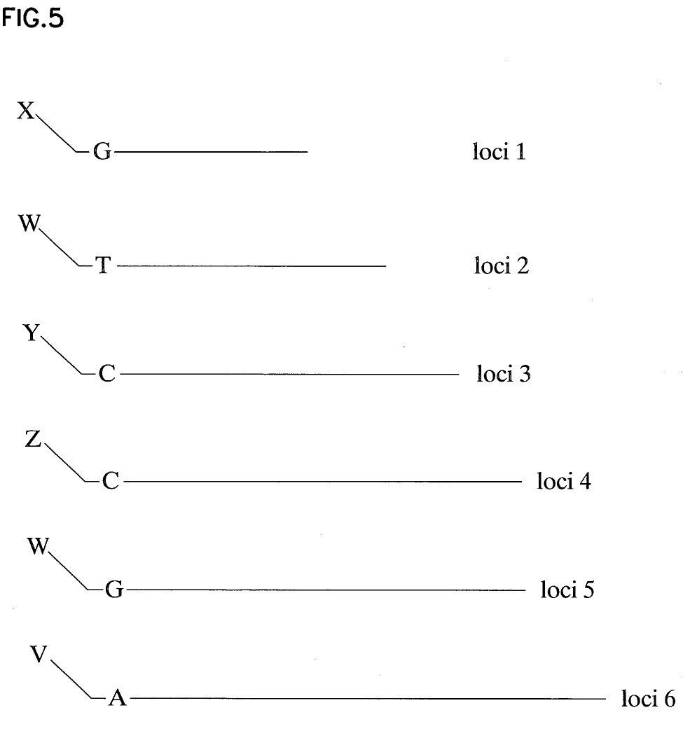

FIG. 5 illustrates schematically the products of the two stage process when applied in a multiplex system;

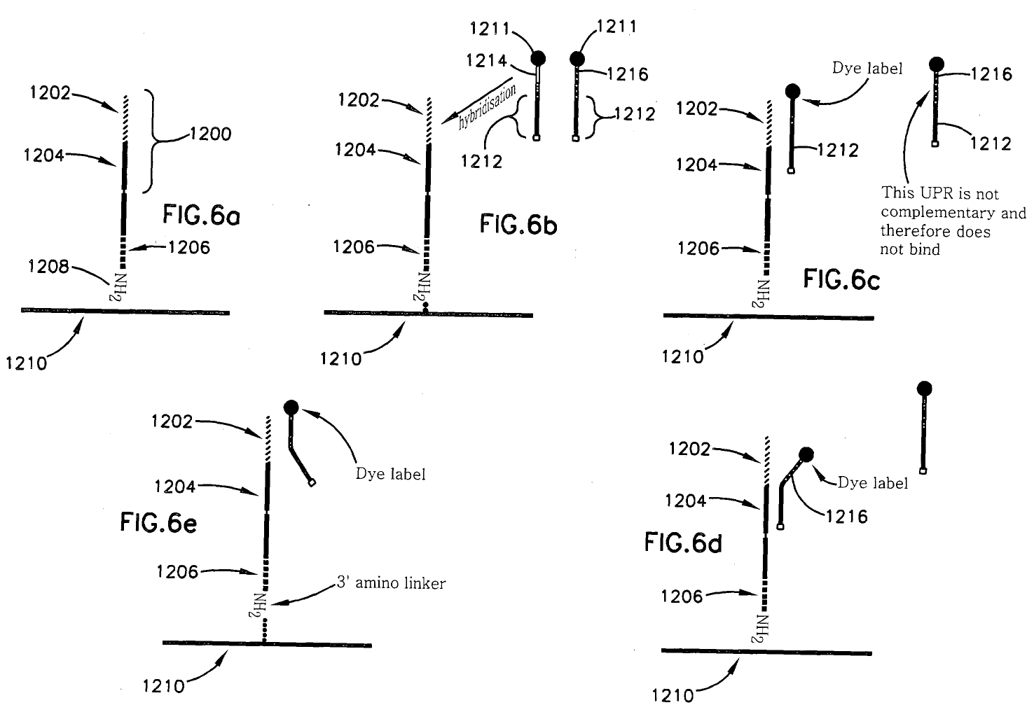

FIG. 6 illustrates an alternative detection process for amplified products prepared according to the present invention;

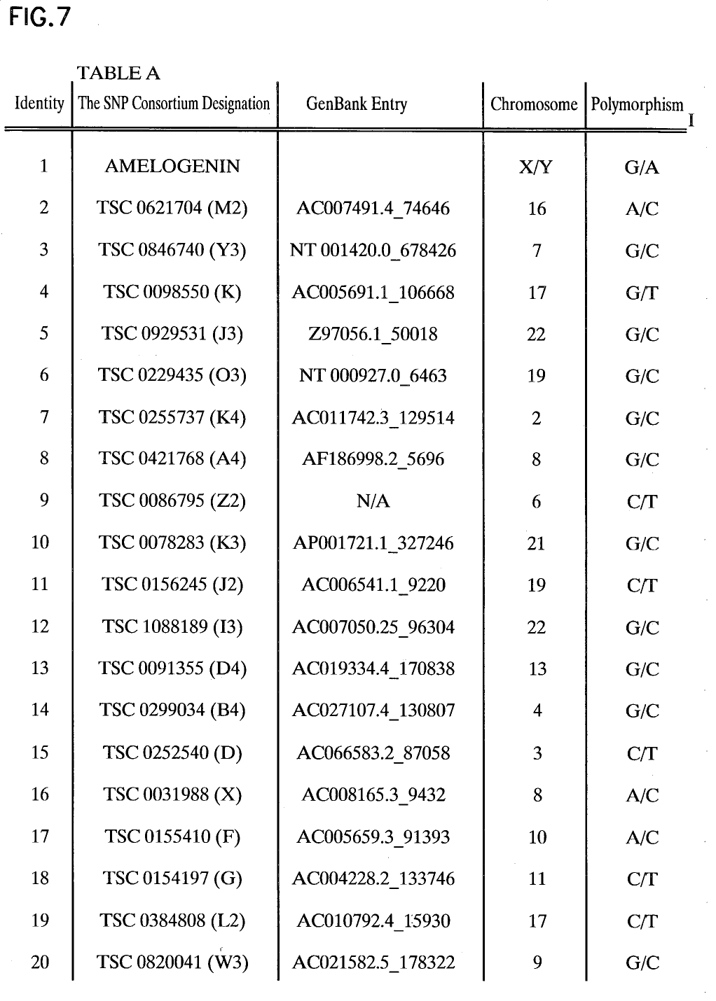

FIG. 7 is a table indicating the SNP consortium designation, provisional gene bank entry, applicable chromosome and applicable polymorphism being considered for each of the specific primers of the present invention in one form;

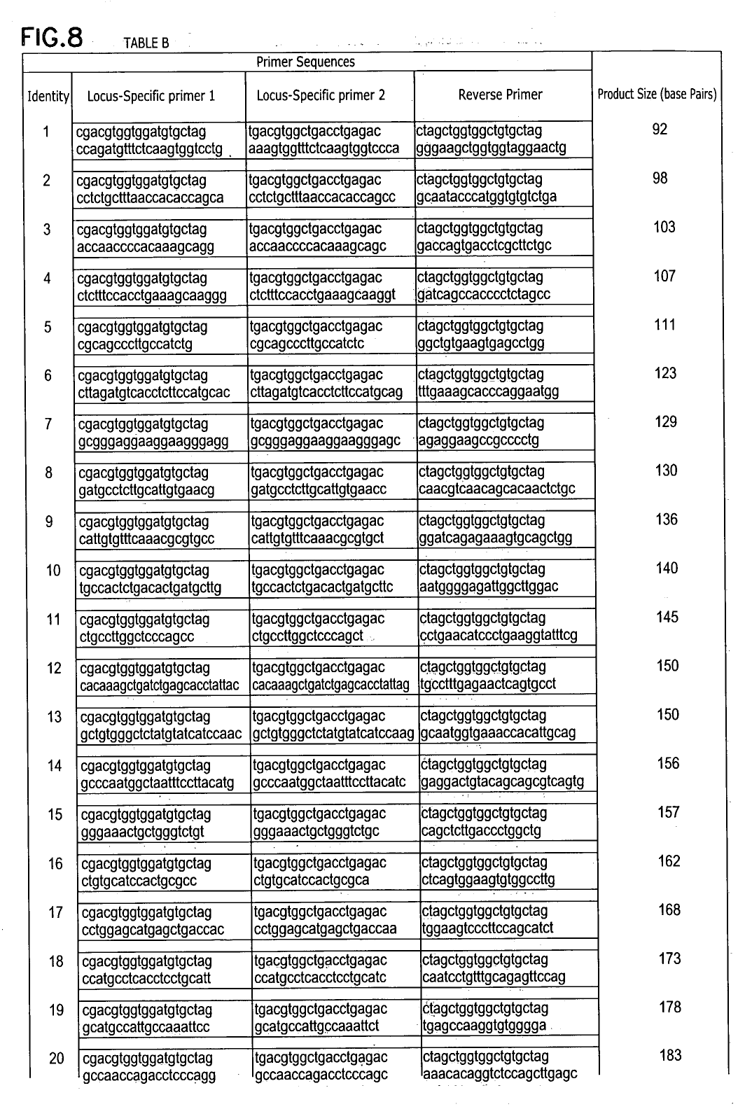

FIG. 8 is a table illustrating the locus specific first primer, locus specific second primer, reverse primer (all written 540 to 340 end) and product size (in base pairs) for each of the specific primers of FIG. 7; and

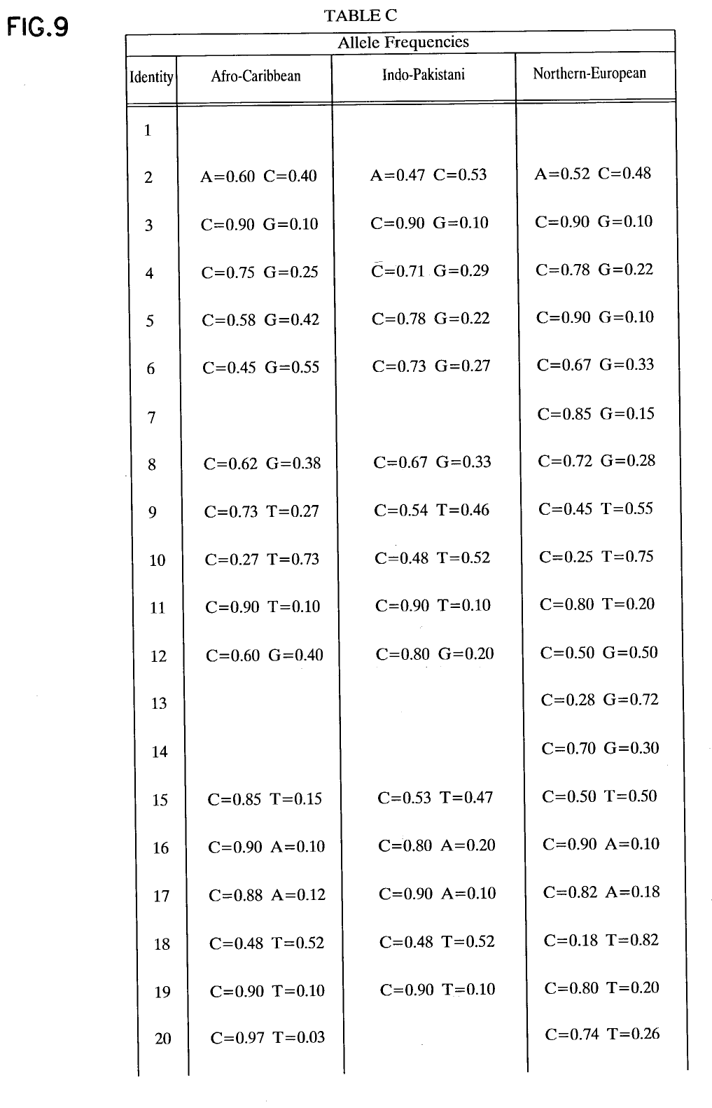

FIG. 9 indicates the allele frequencies for each of the specific primers of FIG. 7 in the context of samples from the Afro-Caribbean, Indo-Pakistani and Northern European ethnic groups;

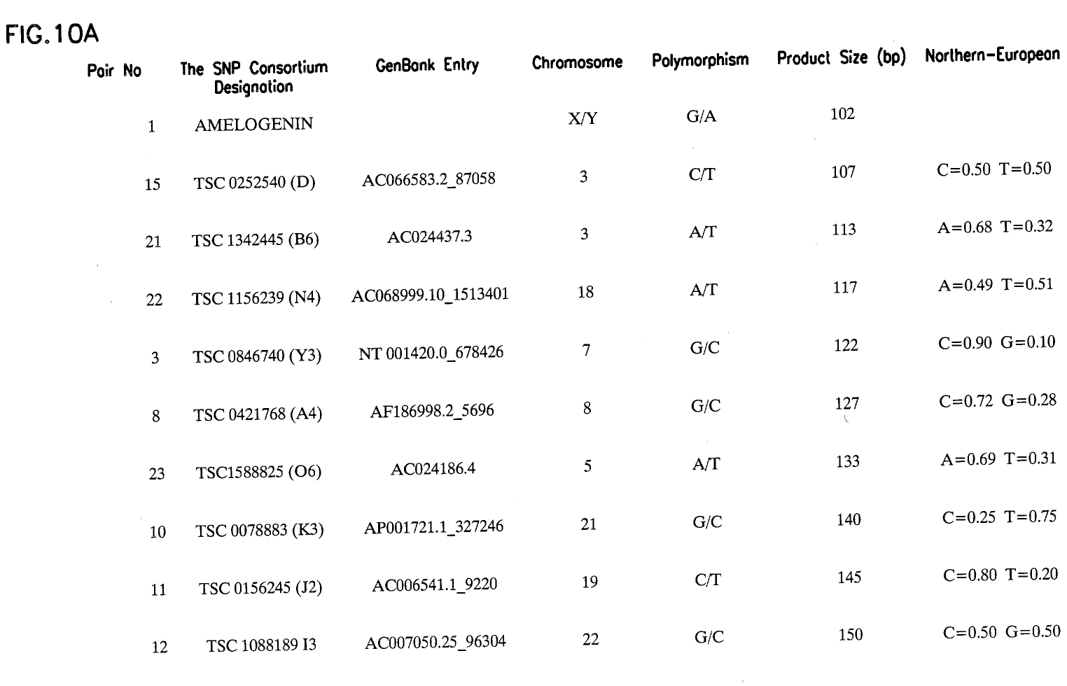

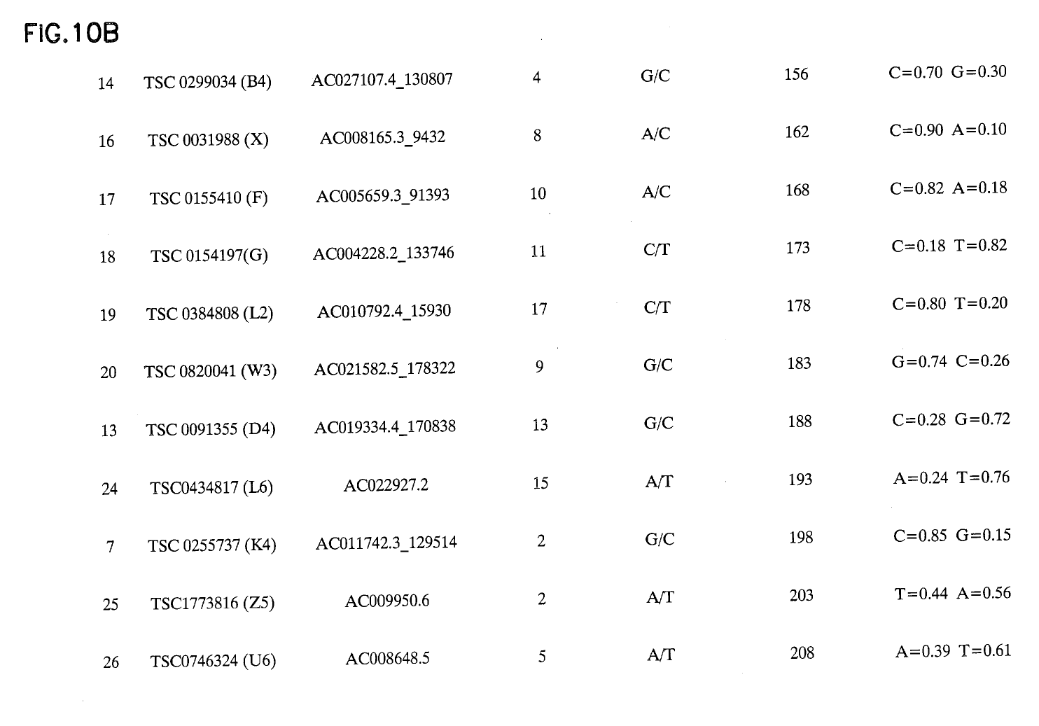

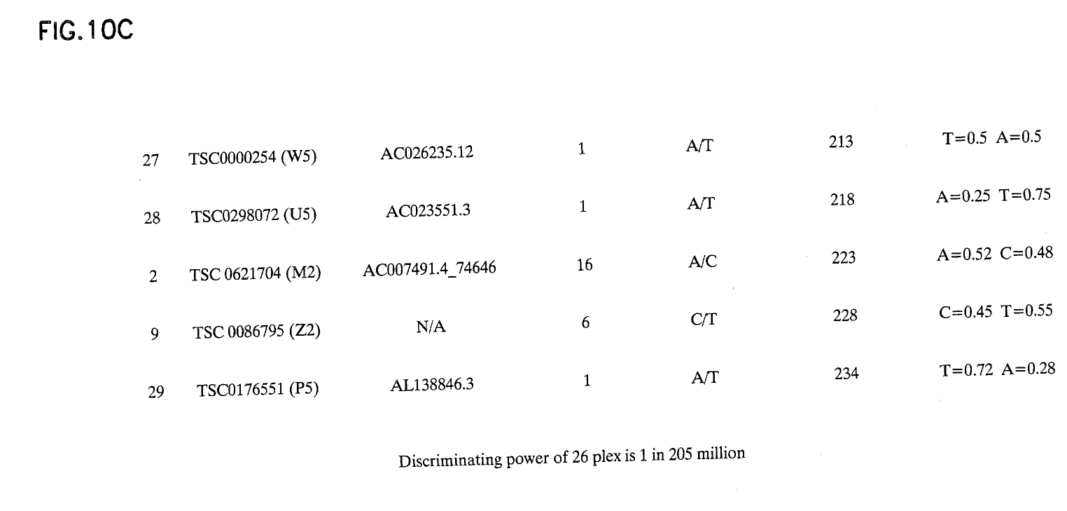

FIG. 10 is a table indicating the SNP consortium designation, provisional GenBank entry, applicable chromosome, applicable polymorphism being considered, product size and allele frequency for the Northern European ethnic group for each of the specific primers of the present invention in another form;

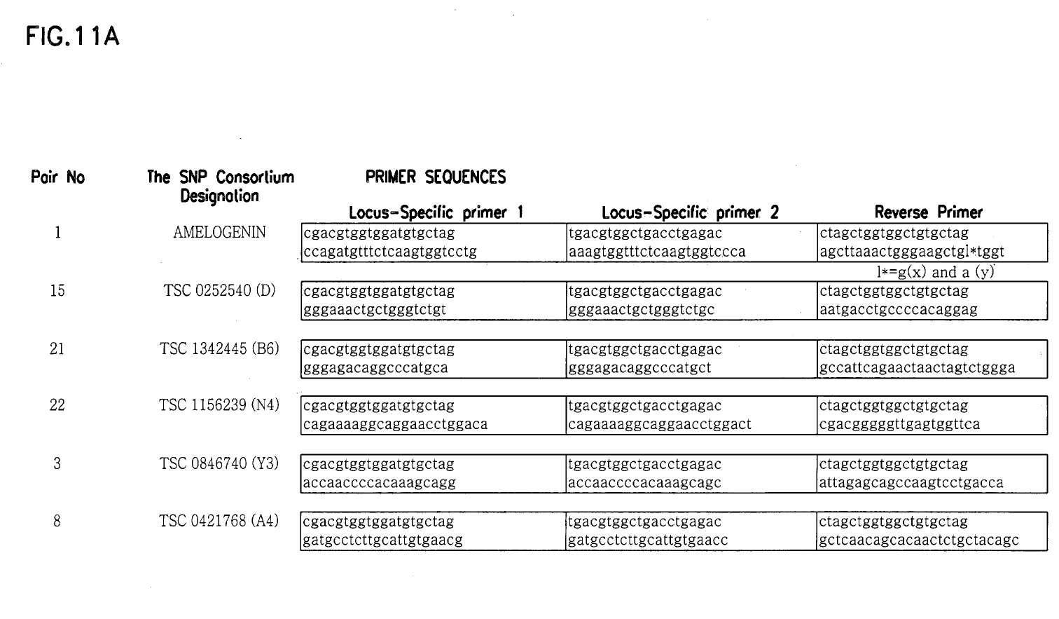

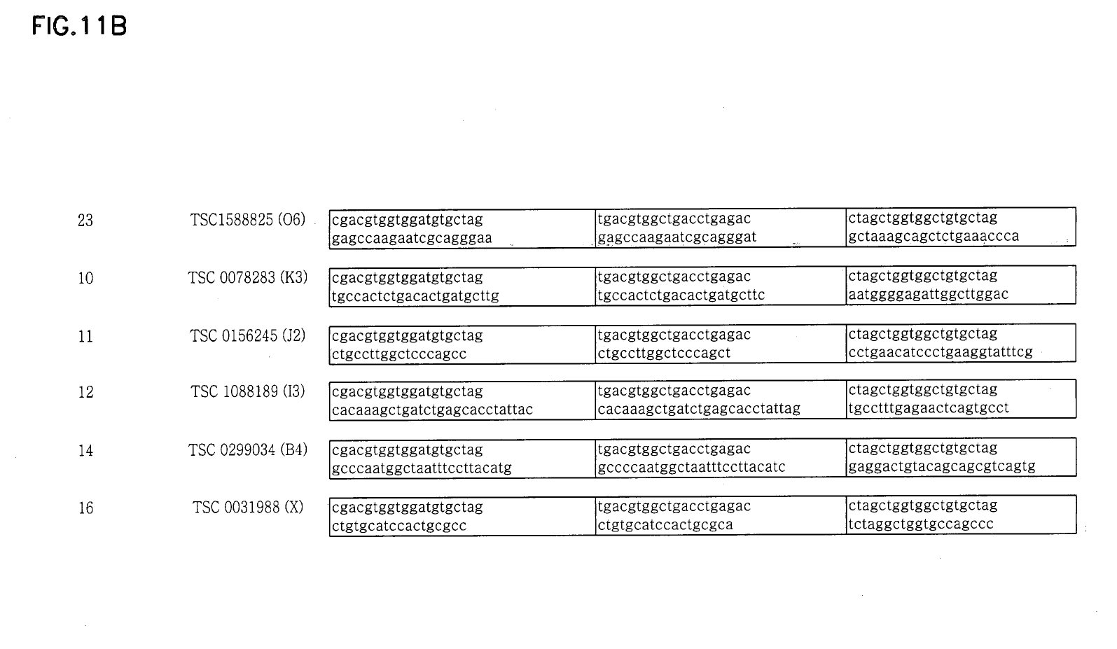

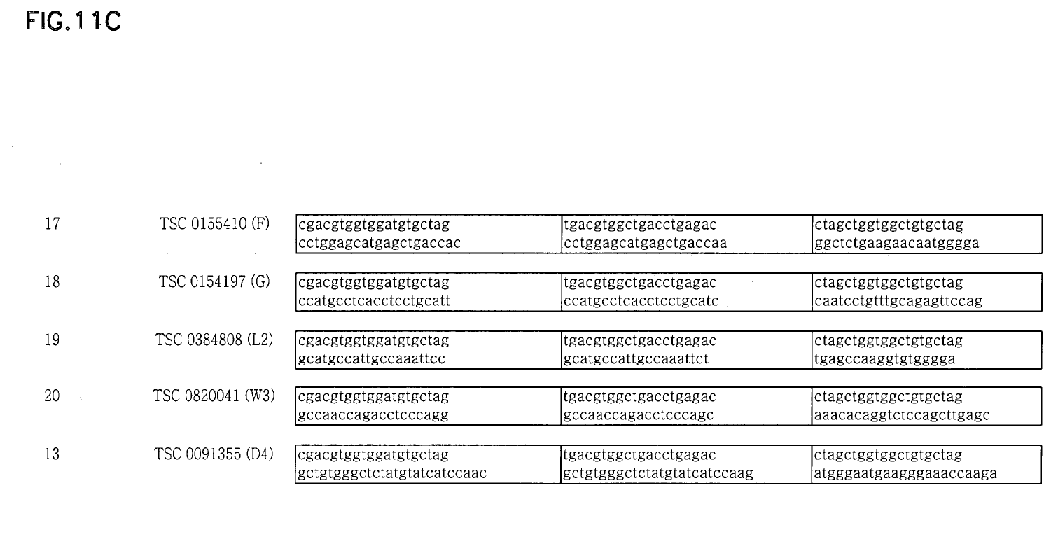

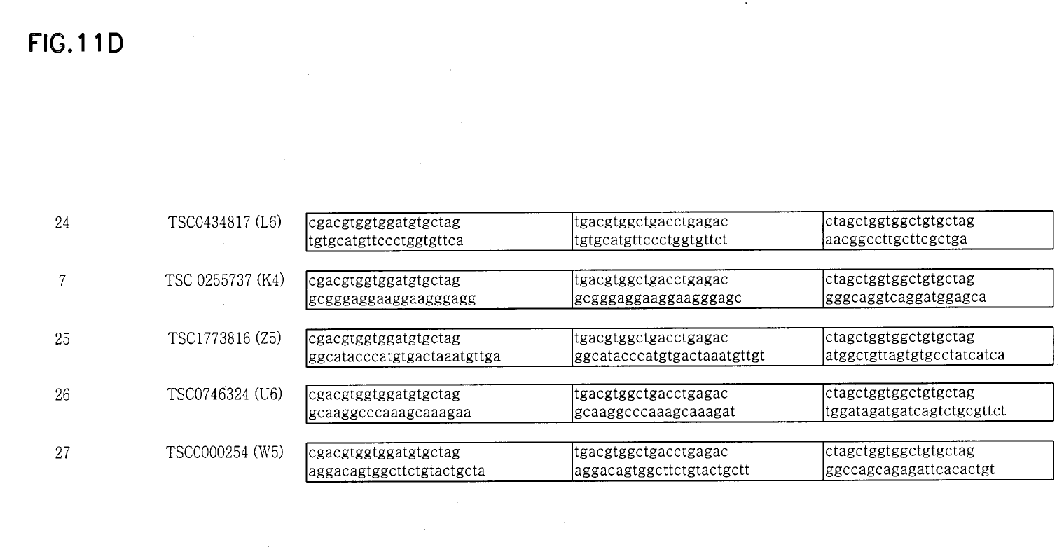

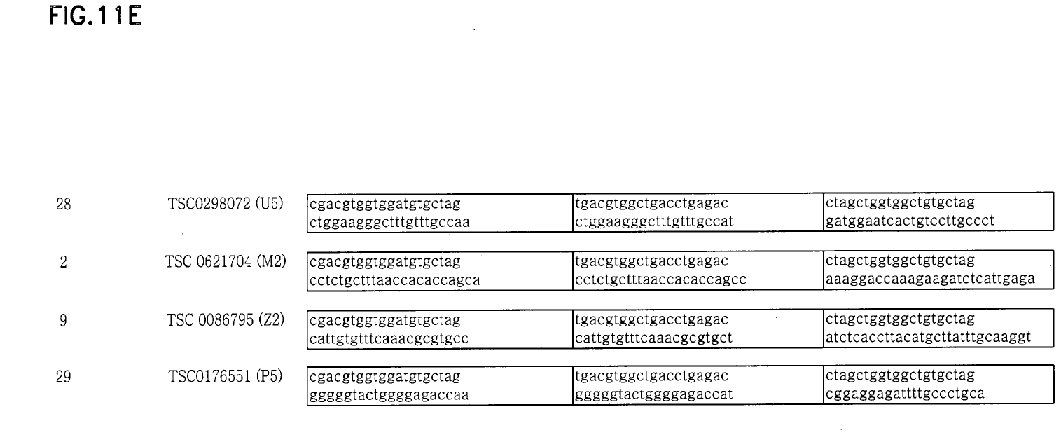

FIG. 11 is a table illustrating the locus specific first primer, locus specific second primer, reverse primer (all written 540 to 340 end) for each of the specific primers of the present invention in the FIG. 10 form.

### BACKGROUND

The nucleotide sequence of humans and other biological entities is in a large part consistent between individuals. Locations are known, however, at which variation occurs. One such form of variation is known as single nucleotide polymorphisms or bi-allelic markers, where the identity of a single nucleotide at a specific location is one of four possibilities from any of the four bases available, A, T, G or C. In many cases the variation is only bi-allelic and hence only one or two possibilities applies. Thus, some individuals may have a sequence incorporating a C base at a particular position, whereas other individuals will have a G base at that position; the surrounding sequences for both individuals being identical.

Medical diagnostics, forensic investigations and other DNA tracing applications make use of such single nucleotide polymorphisms (SNP's) for identification purposes. As the variation between individuals can only be between one of two options, a very substantial number of such locations, loci, must be considered for a statistically significant result, for instance the statistical significance of a match between a collected sample and an individual's makeup to be obtained.

Investigating such a large number of loci, frequently several hundred, on an individual basis is extremely time consuming. To reduce the time taken, it might be desirable to construct multiplexes which allow a substantial number of loci to be investigated simultaneously based on PCR or other amplifying techniques. The design of reliable constructs for a large number of loci, however, is extremely difficult due to problems in interactions between the primers needed for the different loci, different conditions for suitably efficiency amplification of the different primers and a variety of other issues.

Preferred Amplification Technique

The preferred underlying technique is based around two amplification stages, generally achieved through PCR, with both of the stages offering specifity in terms of the SNP's identified and amplified. The two amplification stages can be conducted separately or preferably simultaneously and the amplification products can be analysed in a variety of ways.

FIG. 1 illustrates, according to one embodiment of the invention, a series of stages involved in the first amplification process based around a target template 1 with a potential C or G single nucleotide polymorphism 3 in one strand 5 of that target template 1. As illustrated in step A, the target template strand 5 of the particular individual under consideration has a C nucleotide at the SNP site 3.

The first step in this amplification stage involves contacting the template target 1 with two different forward primers 7 and 9, and a reverse primer 11. The forward primers 7 and 9 are locus specific primers, described in more detail below.

Forward locus specific primer 7 is terminated by a G nucleotide thus rendering it a match with the C nucleotide at the SNP site 3 and resulting in annealing of that primer 7 with the strand 5. The reverse primer 11 is non-specific and anneals to the other strand 13 of the template 1 at the appropriate location.

In step B, the specific forward primer 7 and the reverse primer 11 extend to produce the strands 14 and 16 through primer extension.

Denaturation of the strands results in the separation of the strands 5, 13 from their respective copied strands 14 and 16. The copied strand 14 only is shown in step C and the illustration of the subsequent steps.

Subsequent primer annealing, step D, is then performed again using the two forward primers 7, 9 and reverse primer 11. As we are considering strand 14 it is the reverse primer 11 which attaches to the strand 14 due to its sequence. The specific forward primer 7 would attach to strand 16, once again annealing in alignment with the site of the SNP 3 in that strand's sequence, not shown.

In subsequent primer extension, stage E, the reverse primer 11 extends the sequence of new strand 18 with the appropriate sequence given the sequence of strand 14, including the extension to produce tail portion 19 which arose as the strand 14 included the tail portion 21 of the forward specific primer 7. Due to the G base in the sequence of strand 14, the new strand 18 includes an opposing C base so as to match the identity of the SNP at site 3 in original strand 5.

Repetition of steps A through E over 20 to 25 cycles produces many millions of copies of sequences incorporating the same SNP identity, SNP repeat and surrounding sequence as the target template 1.

FIGS. 2a and b illustrate two locus specific forward primers, suitable for use in the stage detailed above, for use in investigating an SNP which could be either G or C. Each of the locus specific forward primers 30 consists of a locus specific portion 32 which has a sequence corresponding to the sequence of the loci under consideration up to the SNP site. The 340 end 34 of the locus specific forward primers ends in a G nucleotide 34a for one of the primers, FIG. 2a, and in a C nucleotide 34b for the other primer, FIG. 2b. Due to this different nucleotide used at the position corresponding to the SNP, then depending upon the identify of the SNP actually encountered, one of the locus specific forward primers will anneal thereto, but the other will not. Thus it is the forward primer of FIG. 2a which anneals to the target in the example of FIG. 1. This selectivity in annealing gives consequential specifity in the subsequent amplification cycles of the first stage.

In addition to the locus specific portion 32 the locus specific forward primer 30 includes a “universal” primer portion 36. The “universal” primer portion 36 consists of a nucleotide sequence which could be identical for each of the two loci specific forward primers, but is preferably different.

Whilst it is the locus specific portion 32 of the forward primers 30 which determines whether a primer anneals or not to the target, in the second and subsequent copying stages of the amplification process of stage 1, primer extension causes copying of the “universal” primer portion 36 of the primer sequence also.

As previously stated the amplification process of the first stage results in a large number of copy sequences, including the SNP identity reflecting nucleotide.

In the second stage of amplification, illustrated in FIG. 3, a further specific amplification process is performed. It is much preferred that the second stage of amplification be conducted in the same vessel as the first, substantially simultaneous with the first amplification process. Such a possibility is described in more detail below.

For this stage, an aliquot of the amplification products from the first stage, described above, are taken and contacted with a pair of “universal” forward primers and a “universal” reverse primer. These “universal” primers are described in more detail below.

In step A, the strands 40 and 42 (copy strands which are equivalent to strands 14, 18 produced in the first stage as illustrated above) produced by the first stage 1 are denaturated and contacted with the two “universal” forward primers 50, 52 and reverse “universal” primer 54.

The two “universal” forward primers differ in terms of their entire sequence (with one corresponding to the universal portion of one of the primers of the first stage and the other corresponding in sequence to the universal portion of the other forward primer of the first stage), and in terms of a dye unit D or other form of label provided on the 5′ end 56. Depending on the forward primer which annealed in the first stage, FIG. 1, one or other of the universal forward primers anneals and brings with it its particular dye.

As the strands 40 and 42 represent the outcome of copies of copies of the originals being taken, unlike strands 14, 18, they both have tail portions 44, 46 respectively which arise from the copying of the “universal” primer portions of the locus specific forward primer and reverse primer in the first stage.

The “universal” primers 50, 52 each have a sequence corresponding to one of the “universal” primer portion 36 of the first stage locus specific primers 30. The nucleotide identity for the “universal” primers 50, 52 is thus different throughout the two primers 50, 52, with one providing one of the options for corresponding to sequence 36 of the first stage primer and the other providing the other sequence.

The sequence of the primers 50, 52 determines whether or not that primer 50, 52 anneals to the tail portion 44 of the strand 42 or not. In the illustrated case, strand 42 carries the SNP nucleotide C at site 63 as this was a copy of the identity of the SNP at site 3 in the original target strand 5. As a consequence the sequence of the tail portion 44 of strand 42 provides an annealing site for “universal” primer 52, but not primer 50 as those sequences match. The reverse primer 54 anneals to the tail portion 46 of strand 40 due to the sequence matching.

Primer extension, step B, results in the production of strand 60 by matching strand 40, including SNP site copy C, and in the production of strand 62, including the match for the SNP, G, by matching strand 42 by the “universal” reverse primer 54 and specific “universal” forward primer 52 respectively.

Thermal denaturation is then used to separate the strands, step C, and from here on strands 60 and 62 only are considered although similar processes apply to the other strands too.

In annealing step D, the specific “universal” forward primer 52 anneals to the tail 64 of strand 60 due to the presence of a matching C nucleotide sequence in strand 60. The reverse primer 54 anneals to the tail portion 66 of the strand 62.

In the further extension step E, the forward primer 52, which brings with it the label D1, extends the sequence of new strand 68, including tail portion 70. The reverse primer 54 extends the sequence of new strand 72, (thereby reproducing the SNP identity at site 74), including tail portion 76. Strand 62 already incorporates the label D1 from its start as the primer 52 in step A

Once again, repeating stages A to E gives substantial amplification of the sequences and produces a great number of sequences label with a dye D1, the dye being selectively taken up as only one primer anneals and thus takes the dye into the sequence with it.

As described above, the second stage of the process uses a pair of “universal” primers on their own, illustrated in FIGS. 4a and 4b. These consist of a portion 80a, 80b having a sequence identical with the “universal” primer portion 36 of one of the locus specific primers 30 in each case. Thus, one “universal” primer 52, FIG. 4a, is provided with a sequence matching the universal portion sequence 36 of FIG. 2a and the other “universal” primer 50, FIG. 4b, is provided with a sequence matching the universal portion sequence 36 of FIG. 2b.

During stage 2 of the process, these “universal” primers will selectively anneal to the amplification products of the first stage depending upon whether the tail portions extended and amplified during that stage incorporates one or either sequence.

The different “universal” forward primers are provided with different labels/markers, in this case a JOE dye label and an FAM dye label respectively. The dye labels are provided at the 5′ end of the forward primer in the second stage of the process. Of course, other different dyes and other forms of marking, such as radio nuclides could be used.

As the lengths of the sequences forming the amplification products at different loci are designed to be of different length, it is possible to separate those amplification products based on their size, for instance, using electrophoretic techniques and thus produce a series of lines on a gel whose colour is indicative of the SNP variation at the particular loci. The colour will be one of the two colours in each case depending on whether the forwarded universal repeat primer with a sequence matching th universal portion of the first or second forwarding primers of the first stage annealed. Despite the use of only two universal reporter primers for the different loci the product size gives separation in that respect. Where the length of the sequence for one loci may be close to another, different dyes for each of the possibilities can be used. This in the example of FIG. 5 loci 1 and 5 may have the same SNP variation but are sufficiently separable for the same dyes to be used. Loci 4 on the other hand is potentially close to loci 5 results and so different dyes for the results of the G, C variation for loci 4 and 5 are used.

In a variation on the amplification technique intended for use where the amplification products will be analysed using micro arrays or other such techniques, the procedures FIGS. 3a through 3e as described above, is performed, but the two universal forward primers are provided within dye or other distinctive units. As a result a very substantial number of copies of the amplification products from the first stage are obtained, providing copies of the SNP, locus specific portion and universal portion.

Determination of the identity of the SNP present is achieved using the technique of FIGS. 6a to 6e as will now be described.

As illustrated in FIG. 6a, the amplification product consists of the forward primer sequence 1200, including the universal primer portion 1202 and locus specific portion 1204 and the reverse primer portion 1206. By providing an amino end group 1208 on the terminal 5′ end of the reverse primer, the strand can be covalently attached to an epoxy-silane treated glass slide, 1210. The preparation of such epoxy-silane slides is based on the method of Beatty et al, Molecular Biology, Volume 4, 1995, 213-225.

Other attachment chemistry, for instance the use of 5′ ends labelled with phosphorothiate can be used to attach such strands to bromo-acetamide slides for instance.

Generally, the amplified product is purified for instance using a centricon filtration to remove any incorporated primer and dNTP's. It is then extracted with water before spotting on to the glass slides, for instance using an Amersham generation 3 micro array spotter. The slides can be incubated in a high humidity chamber for between 30 minutes and 2 hours at 20 °C. to 40 °C. before washing in water at 95 °C., 10 mM trithyomine (pH 9.2) at room temperature, and two further washes with the water at 60 °C. before being stored dry at room temperature.

To affect the detection step, the single strands must be contacted with suitable fluorescent probe constructs. In FIG. 6b a biallelic system is being introduced, and as a consequence two different fluorescent probes are introduced. Each fluorescent probe has a different dye label 1211, common locus specific portion 1212 and different universal portions 1214 and 1216 respectively, which different universal portions correspond to the sequence used in one of the further portions and second further portions of the first and second sets of primers discussed above. Hybridisation is carried out at low temperature, around 40 °C. Probe specifity is controlled by carrying out post-hybridisation washes at higher temperatures.

As illustrated in FIG. 6c only one of the fluorescent probes is sufficient complimentary to hybridise completely to the fixed strand. By conducting the post-hybridisation washes at a sufficiently high temperature (60 °C. to 75 °C.) specifity of the probe is maintained as at such temperatures the fact that the locus specific portion 1212 of the other fluorescent probe corresponds insufficiently to remain hybridised due to the difference between the universal primer portion 1202 and 1216, FIG. 6d. As a consequence the second fluorescent dye label is not retained by the single strand.

In a similar basis, FIG. 6e, if the universal primer portions are complimentary, but the locus specific region is different, a fluorescent probe for a different locus, hybridisation cannot occur sufficiently for the fluorescent probe to be retained on a similar basis.

Improved Primers, Combinations and Multiplexes

To improve further on this underlying technique a number of optimal loci targets have been identified and optimised locus specific primers for investigating them, with a view to determining the SNP present, have been designed. The loci and locus specific primers for investigating them have also been optimised in a combined sense to render them suitable for use in combination with one another and particularly in a multiplex involving a number of such primers alone and/or potentially in combination with other primers. The end result is a set up offering rapid analysis in an effective manner which functions on small starting samples.

The loci selected have been evaluated to ensure, in each case, that the SNP, and more particularly the surrounding sequence to the SNP, is suited to the design of primers for its amplification. In general, the determination involves establishing the melting temperature of a primer which abuts the side of the locus, the length of primer necessary to anneal to that side effectively, and the balance between the two. In the present case, the applicant has determined that a melting temperature, Tm, of around 60 °C. and primer lengths of around 20 bases are preferred. As such loci and primers for them having such features have been sought. The initial consideration of each locus also includes an evaluation of how AT rich the sequence is around the SNP site, as such sequences necessitate very long primers to give effective annealing. Loci involving such AT rich sequences are generally avoided in the present invention's optimised performance. Whilst the overall primer sequence is considered in this evaluation, it is the locus specific portion (which will actually hybridise in practice in the case of the primer with the pairing base to the SNP) which is particularly considered against these criteria.

Suitable target loci then have primer pairs generated for them with the initial part of the each forward primer sequence, the locus specific portion, matching the DNA sequence adjacent to the SNP site on its 3′ end side. The primer also includes a second part namely the “universal” primer portion which consists of a nucleotide sequence which is identical for each of the two loci specific forward primer in a primer pair, save for a single nucleotide location at the junction between the “universal” primer portion and the locus specific portion. This nucleotide commonly has an equivalent identity to the 3′ end base of the locus specific portion primer in each case. Thus one primer of the pair might have a 3′ end which is a G nucleotide, and with a G nucleotide linking the locus specific portion and the universal primer portion together, whereas the other primer of that pair has a C nucleotide present. A single reverse primer is also needed for each pair and this has a sequence which matches the DNA sequence of the other strand at a site commencing within 2 to 120 bases of the 3′ end side.

Each locus specific portion is intended only to bind to a DNA sequence incorporating the matching base to its 3′ end at the SNP site. The other primer in the pair does not bind because its locus specific portion is not a complete match.

The universal primer portions are intended not to prime human DNA (thus artefacts mediated by mis-priming of degraded DNA are minimal, and the pull up artefact is easily recognised since the electrophoretic migration rates of different SNP's are different); and have different sequences from one another within a primer pair and between different primers in the multiplex.

Providing locus specific portions and universal primer portions according to these criteria gives an optimised form.

Once the primer pairs for each locus have been constructed the loci are evaluated to ensure optimised performance and in particular to ensure they do not face any problems. Situations in which the SNP is actually established to be monomorphic and/or where it is established that there are actually copies of the SNP and its surrounding sequence are present on the genome lead to the loci and constructed primer pair being discarded from the optimised form.

The loci are also carefully evaluated to ensure that the SNP variation for that loci within the population as a whole and particularly within a number of subsets of the population corresponding to major ethnic groupings is suitable for forensic purposes. An SNP polymorphism providing the frequency of an allele between 0.1 and 0.9 in each of the three principal ethnic groups (Caucasian, Asian, Afro-Caribbean) is preferred for this purpose. This contrasts markedly with SNP's selected for use as medical condition indicators where unusual and rare SNP polymorphism are sought. Such SNP's are discounted from being optimised targets in the present case as these are unsuited to forensic purposes.

The particular primers are also considered and optimised to give good amplification which is of balanced efficiency and which avoids the amplification of artefacts. Unbalanced performance between different primer pairs gives different levels of amplified product for the different loci and interpretation problems as a result.

The primers are also designed to ensure that the amplification products are of different sizes to one another so that they can be separated in the eventual analysis process whether this be gel electrophoresis, capillary array electrophoresis or other technique.

Optimised targets in terms of loci determined according to these principals are identified by their SNP consortium designation (as provided at a date of Aug. 20, 2001) and/or GenBank entry in attached FIG. 7 (as Table A or FIG. 10 as Table D). The chromosome number and polymorphism variation which can occur at that target is also stated in these tables.

In FIG. 8, Table B and FIG. 11, Table E provides the sequences for a pair of forward primers and a reverse primer of an optimised type for investigating the particular targets specified are provided. The sequence is written from the 5′ end to the 3′ end. The sequences are split into two lines with the top line being the locus specific portion sequence and the lower line the universal primer portion sequence (in practice the sequence is continuous). The primer sequence overall size is also listed in FIG. 8 and FIG. 11.

The frequency of occurrence for each of the two possible alleles for each locus, for the three principal ethnic groups (Afro-Caribbean, Indo-Pakistani and Northern European), is set out in FIG. 9, Table C and FIG. 10, Table D (for Northern European). In each case it is apparent that the optimised targets indicate the variation as being 0.1 and 0.9. This ensures a significant variation between profiles for normal individuals and renders the targets as being suited for forensic purposes. The targets considered in medical diagnostic tests are ones in which one of the polymorphism identities only occurs very rarely, and hence has a strong association with the medical condition of interest when it occurs.

Optimised multiplexes are generally constructed from 10 candidate SNP's by selecting the best balance. Melting temperature, amplification efficiency and balancing amplification between different primers from amongst these already optimised candidates gives still further optimised multiplexes. The selection is made so that no two primer pairs have the same size and would hence give amplification products of the same size. Products differing by 5 bases or more are preferred. Around 10 primer identities for each multiplex is preferred, although different numbers can be deployed.

Tables 7, 8 and 9 represent an initial optimised set of primers from which primers can be selected to form an optimised multiplex. FIGS. 10 and 11 set out details of a still further optimised set of primers from which primers can be selected to give still further improved multiplexes. Some primer pairs and primers common to both sets of primers occur.

Experimental Procedure Example

Mastermix

For convenience a “Mastermix” can be employed. The Mastermix contains the following constituents:

| Constituent | Conc. In final 25 μl reaction vol. |
|---|---|
| Perkin/Elmer PCR Buffer II | x1 |
| Mg²⁺ ions | 1.5 mM |
| dNTPs | 225 μM |
| Universal Primer A - FAM labelled | 2 μM |
| Universal Primer B - JOE labelled | 2 μM |
| Bovine Serum Albumin | 0.4 μg/ul |

The mastermix should be stored frozen at −20 °C. and should not be subjected to repeat freeze-thaw cycles. The buffer requirements of the PCR are supplied by the mastermix therefore the primers should be constituted in distilled H₂O.

Initially the final concentration of the forward locus-specific primers in the final reaction volume of 25 μl should be 50 nM and the concentration of the reverse locus primers should be 100 nM. These concentrations can be varied if necessary according to the peaks produced at each locus. An increase in the concentration of a reverse locus primer results in an increase in the quantity of both allele products. An increase in one of the forward locus primers results in an increase in the quantity of that particular allele product only. Primers should be HPLC purified and a stock concentration of 10 μM is convenient. It is critical that locus-specific primers are not subjected to freeze-thaw cycles as this will seriously affect the sensitivity of the system.

DNA Polymerase

The technique has been developed using Perkin/Elmer TAQ-Gold DNA polymerase. A TAW concentration of 5.0U/25 μl reaction vol. is required.

Template DNA

The technique requires 0.5 ng-1.0 ng of template DNA in a final reaction volume of 25 μl. For a final concentration of 1.0 ng/25 μl, 4 μl of a 0.25 ng/μl DNA extract is used in the 25 μl reaction volume. DNA samples isolated by various techniques are all suitable.

The constituents of a typical reaction mix for the determination of a 5-locus SNP pentaplex are given in the following table:

Constituents of a 5-Locus SNP Pentaplex

| Constituent | Volume | |
|---|---|---|
| Mastermix* | 255 | μl |
| 5 × Locus-specific forward primers (A) [10 μM] | 5 × 5 | μl |
| 5 × Locus-specific forward primers (B) [10 μM] | 5 × 5 | μl |
| 5 × Locus-specific reverse primers (C) [10 μM] | 5 × 10 | μl |
| TAQ-Gold | 40 | μl |
| H₂O | 445 | μl |
| Total | 840 | μl |

*please note that this volume is dependent on the stock concentration of the constituents used to make up the mastermix. 255 μl is an example volume.

21 μl of the above reaction mix are added to 4 μl of DNA extract (0.25 ng/μlDNA)/Control Sample to give a final reaction volume of 25 μl. The above mix is sufficient for 40 tests.

Additional loci may be studied and will involve the addition of further primers of the reaction mix. In this case the volume of H₂O is adjusted such that the final volume of the reaction mix remains at 840 μl.

Cycling Conditions

The cycling conditions employed are shown below:

| Taq-Gold | | |
|---|---|---|
| Activation | | 95° C. for 11 min |
| Phase 1 | 7-Sep PCR Cycle | Denature at 94° C. for 30 s |
| | | Anneal at 60° C. for 15 s |
| | | Extension at 72° C. for 15 s |
| | | Anneal at 60° C. for 15 s |
| | | Extension at 72° C. for 15 s |
| | | Anneal at 60° C. for 15 s |
| | | Extension at 72° C. for 30 s |

2 Repeats

| | | |
|---|---|---|
| Phase 2 | 2-Step PCR Cycle | Denature at 94° C. for 30 s |
| | | Anneal/Extension at 76° C. for 1 min 45 s |

33 Repeats

| | | |
|---|---|---|
| Phase 3 | 3-Step PCR Cycle | Denature at 94° C. for 1 min |
| | | Anneal at 60° C. for 30 s |
| | | Extension at 72° C. for 1 min |

3 Repeats
Phase 1 During this phase only the locus-specific regions of the “URP-tagged” primers should anneal to template DNA. The presence of a polymorphism at the SNP site on the original template DNA results in the formation of 2 “target amplicons” which bear different universal sequences at one of the strands of the amplicon. There are only two cycles during this phase and thus the synthesis of non-specific products is minimised.
Phase 2 - Cycle Steps A 2-step PCR cycle with a combined annealing/extension step at 76° C. is employed during the 31 cycles of phase 2. At this temperature only the locus-specific primers are able to function by virtue of their greater length (approximately 40 bases) and consequently higher Tm values. Amplification by the shorter, labelled universal primers is inhibited as is the production of non-specific PCR products. The production of labelled products during
#this phase is thus minimum. Amplification by the long, locus-specific primers at an annealing/extension temperature of 76° C. is thought to be highly specific but inefficient. This inefficiency results in the requirement for a larger number of cycles during this phase.
Phase 3 During these final three cycles production of fluorescently-labelled products via amplification by labelled universal reporter primer is permitted by a reduction in the annealing temperature to 60° C. Amplicons are produced which are labelled with one of two fluorescent dyes. The fluorescent label which is present at an amplicon is indicative of
#the base which was present at the SNP site on the original template DNA. Cycling may be performed with a Perkin/Elmer 9600 thermal cycler or a MJ Instruments Tetrad Engine thermal cycler.

Gel Electrophoresis

The products of PCR amplification are separated using a polyacrylamide denaturing gel (0.2 mm thickness) run on a Perkin/Elmer 377 gel sequencer. An internal size ladder is employed and this is added to loading buffer (dextran blue/formamide) at a ratio of 1 vol Perkin/Elmer Rox Standard: 7 vol of dextran blue/formamide loading buffer. PCR samples (2 μl) are mixed with an equal volume of loading buffer (containing Rox standard), denatured at 90 °C. for 2 min on a thermal cycler, rapidly cooled by placing immediately on ice and then 2 μl is loaded onto the gel (36-lane comb). Filter wheel setting F with an F matrix is employed for the electrophoresis run.

Products labelled with FAM-dye appear blue and products labelled with JOE-dye appear green. ROX size standard fragments appear red.

The products of particular loci are identified by product size and the alleles present at a particular locus are indicated by the colour of the product. Homozygotes are indicated by the presence of a single product (appearing either blue or green) at a particular locus. Both products (blue and green) are present from heterozygotes.

Artificial “pull-up” peaks which appear to indicate the presence of a heterozygote can be readily distinguished from true dual products by a careful examination of the electrophoretogram. “Pull-Up” peaks lie immediately beneath a larger peak of a different colour and have the same scan numbers for the peak maxima. The peaks from a true heterozygote will be slightly mis-aligned and will not show the same scan numbers for the peak maxima. This is due to the presence of the different due labels and the different 20-base universal sequences present on the products of different alleles.

1. A mixture including at least one primer pair, at least one of those primer pairs being a primer pair having or including one of the following sequences:

| | | | |
|---|---|---|---|
| Pair 1 | Primer 1 | cgacgtggtggatgtgctagccagatgtttctcaagtggtcctg | SEQ ID NO 1 |
| | Primer 2 | tgacgtggctgacctgagacaaagtggtttctcaagtggtccca | SEQ ID NO 2 |
| Pair 2 | Primer 1 | cgacgtggtggatgtgctagcctctgctttaaccacaccagca | SEQ ID NO 4 |
| | Primer 2 | tgacgtggctgacctgagaccctctgctttaaccacaccagcc | SEQ ID NO 5 |
| Pair 3 | Primer 1 | cgacgtggtggatgtgctagaccaaccccacaaagcagg | SEQ ID NO 7 |
| | Primer 2 | tgacgtggctgacetgagacaccaaccccacaaagcagc | SEQ ID NO 8 |
| Pair 4 | Primer 1 | cgacgtggtggatgtgctagctctttccacctgaaagcaaggg | SEQ ID NO 10 |
| | Primer 2 | tgacgtggctgacctgagacctctttccacctgaaagcaaggt | SEQ ID NO 11 |
| Pair 5 | Primer 1 | cgacgtggtggatgtgetagcgcagcccttgccatctg | SEQ ID NO 13 |
| | Primer 2 | tgacgtggctgacctgagaccgcagcccttgccatctc | SEQ ID NO 14 |
| Pair 6 | Primer 1 | cgacgtggtggatgtgctagcttagatgtcacctcttccatgcac | SEQ ID NO 16 |
| | Primer 2 | tgacgtggctgacctgagaccttagatgtcacctcttccatgcag | SEQ ID NO 17 |
| Pair 7 | Primer 1 | cgacgtggtggatgtgctaggcgggaggaaggaagggagg | SEQ ID NO 19 |
| | Primer 2 | tgacgtggctgacctgagacgcgggaggaaggaagggagc | SEQ ID NO 20 |
| Pair 8 | Primer 1 | cgacgtggtggatgtgctaggatgcctcttgcattgtgaacg | SEQ ID NO 22 |
| | Primer 2 | tgacgtggctgacctgagacgatgcctcttgcattgtgaacc | SEQ ID NO 23 |
| Pair 9 | Primer 1 | cgacgtggtggatgtgctagcattgtgtttcaaacgcgtgcc | SEQ ID NO 25 |
| | Primer 2 | tgacgtggctgacctgagaccattgtgtttcaaacgcgtgct | SEQ ID NO 26 |
| Pair 10 | Primer 1 | cgacgtggtggatgtgctagtgccactctgacactgatgcttg | SEQ ID NO 28 |
| | Primer 2 | tgacgtggctgacctgagactgccactctgacactgatgcttc | SEQ ID NO 29 |
| Pair 11 | Primer 1 | cgacgtggtggatgtgctagctgccttggctcccagcc | SEQ ID NO 31 |
| | Primer 2 | tgacgtggctgacctgagacctgccttggctcccagct | SEQ ID NO 32 |
| Pair 12 | Primer 1 | cgacgtggtggatgtgctagcacaaagctgatctgagcacctattac | SEQ ID NO 34 |
| | Primer 2 | tgacgtggctgacctgagaccacaaagctgatctgagcacctattag | SEQ ID NO 35 |
| Pair 13 | Primer 1 | cgacgtggtggatgtgctaggctgtgggctctatgtatcatccaac | SEQ ID NO 37 |
| | Primer 2 | tgacgtggctgacctgagacgctgtgggctctatgtatcatccaag | SEQ ID NO 38 |
| Pair 14 | Primer 1 | cgacgtggtggatgtgctaggcccaatggctaatttccttacatg | SEQ ID NO 40 |
| | Primer 2 | tgacgtggctgacctgagacgcccaatggctaatttccttacatc | SEQ ID NO 41 |
| Pair 15 | Primer 1 | cgacgtggtggatgtgctaggggaaactgctgggtctgt | SEQ ID NO 43 |
| | Primer 2 | tgacgtggctgacctgagacgggaaactgctgggtctgc | SEQ ID NO 44 |
| Pair 16 | Primer 1 | cgacgtggtggatgtgctagctgtgcatccactgcgcc | SEQ ID NO 46 |
| | Primer 2 | tgacgtggctgacctgagacctgtgcatccactgcgca | SEQ ID NO 47 |
| Pair 17 | Primer 1 | cgacgtggtggatgtgctagcctggagcatgagctgaccac | SEQ ID NO 49 |
| | Primer 2 | tgacgtggctgacctgagaccctggagcatgagctgaccaa | SEQ ID NO 50 |
| Pair 18 | Primer 1 | cgacgtggtggatgtgctagccatgcctcacctcctgcatt | SEQ ID NO 52 |
| | Primer 2 | tgacgtggctgacctgagacccatgcctcacctcctgcatc | SEQ ID NO 53 |
| Pair 19 | Primer 1 | cgacgtggtggatgtgctaggcatgccattgccaaattcc | SEQ ID NO 55 |
| | Primer 2 | tgacgtggctgacctgagacgcatgccattgccaaattct | SEQ ID NO 56 |
| Pair 20 | Primer 1 | cgacgtggtggatgtgctaggccaaccagacctcccagg | SEQ ID NO 58 |
| | Primer 2 | tgacgtggctgacctgagacgccaaccagacctcccagc | SEQ ID NO 59 |
| Pair 21 | Primer 1 | cgacgtggtggatgtgctaggggagacaggcccatgca | SEQ ID NO 61 |
| | Primer 2 | tgacgtggctgacctgagacgggagacaggcccatgct | SEQ ID NO 62 |
| Pair 22 | Primer 1 | cgacgtggtggatgtgctagcagaaaaggcaggaacctggaca | SEQ ID NO 64 |
| | Primer 2 | tgacgtggctgacctgagaccagaaaaggcaggaacctggact | SEQ ID NO 65 |
| Pair 23 | Primer 1 | cgacgtggtggatgtgctaggagccaagaatcgcagggaa | SEQ ID NO 67 |
| | Primer 2 | tgacgtggctgacctgagacgagccaagaatcgcagggat | SEQ ID NO 68 |
| Pair 24 | Primer 1 | cgacgtggtggatgtgctagtgtgcatgttccctggtgttca | SEQ ID NO 70 |
| | Primer 2 | tgacgtggctgacctgagactgtgcatgttccctggtgttct | SEQ ID NO 71 |
| Pair 25 | Primer 1 | cgacgtggtggatgtgctagggcatacccatgtgactaaatgttga | SEQ ID NO 73 |
| | Primer 2 | tgacgtggctgacctgagacggcatacccatgtgactaaatgttgt | SEQ ID NO 74 |
| Pair 26 | Primer 1 | cgacgtggtggatgtgctaggcaaggcccaaagcaaagaa | SEQ ID NO 76 |
| | Primer 2 | tgacgtggctgacctgagacgcaaggcccaaagcaaagat | SEQ ID NO 77 |
| Pair 27 | Primer 1 | cgacgtggtggatgtgctagaggacagtggcttctgtactgcta | SEQ ID NO 79 |
| | Primer 2 | tgacgtggctgacctgagacaggacagtggcttctgtactgctt | SEQ ID NO 80 |
| Pair 28 | Primer 1 | cgacgtggtggatgtgctagctggaagggctttgtttgccaa | SEQ ID NO 82 |
| | Primer 2 | tgacgtggctgacctgagacctggaagggctttgtttgccat | SEQ ID NO 83 |
| Pair 29 | Primer 1 | cgacgtggtggatgtgctaggggggtactggggagaccaa | SEQ ID NO 85 |
| | Primer 2 | tgacgtggctgacctgagacgggggtactggggagaccat | SEQ ID NO 86 |

or sequences at least 75% homologous thereto.

2. A mixture according to claim 1 which includes at least 8 primer pairs having one or both of the sequences listed for the pairs of claim 1.

3. A mixture according to claim 1 or 2 which includes at least 8 primer pairs, from the listed primer pairs of claim 1.

4. A mixture according to any of claims 1 to 3 in which the mixture includes at least one primer pair, at least one of those primer pairs being a primer pair having or including one of the sequences of a primer pair selected from pair 1, pair 2, pair 3, pair 7, pair 8, pair 9, pair 10, pair 11, pair 12, pair 13, pair 14, pair 15, pair 16, pair 17, pair 18, pair 19, pair 20, pair 21, pair 22, pair 23, pair 24, pair 25, pair 26, pair 27, pair 28, pair 29 as provided in claim 1.

5. A mixture according to any preceding claim in which the primers of a pair have a locus specific portion and a universal portion.

6. A mixture according to any preceding claim in which one or more of the listed sequences of the primers have locus specific portions and/or universal portions according to the following forms:

| | | | | |
|---|---|---|---|---|
| Pair 1 | Primer 1 | cgacgtggtggatgtgctag (universal portion) | | |
| | | | {close oversize parenthesis} | SEQ ID NO 1 |
| | | ccagatgtttctcaagtggtcctg (locus specific portion) | | |
| | Primer 2 | tgacgtggctgacctgagac (universal portion) | | |
| | | | {close oversize parenthesis} | SEQ ID NO 2 |
| | | aaagtggtttctcaagtggtccca (locus specific portion) | | |
| Pair 2 | Primer 1 | cgacgtggtggatgtgctag (universal portion) | | |
| | | | {close oversize parenthesis} | SEQ ID NO 4 |
| | | cctctgctttaaccacaccagca (locus specific portion) | | |
| | Primer 2 | tgacgtggctgacctgagac (universal portion) | | |
| | | | {close oversize parenthesis} | SEQ ID NO 5 |
| | | cctctgctttaaccacaccagcc (locus specific portion) | | |
| Pair 3 | Primer 1 | cgacgtggtggatgtgctag (universal portion) | | |
| | | | {close oversize parenthesis} | SEQ ID NO 7 |
| | | accaaccccacaaagcagg (locus specific portion) | | |
| | Primer 2 | tgacgtggctgacctgagac (universal portion) | | |
| | | | {close oversize parenthesis} | SEQ ID NO 8 |
| | | accaaccccacaaagcagc (locus specific portion) | | |
| Pair 4 | Primer 1 | cgacgtggtggatgtgctag (universal portion) | | |
| | | | {close oversize parenthesis} | SEQ ID NO 10 |
| | | ctctttccacctgaaagcaaggg (locus specific portion) | | |
| | Primer 2 | tgacgtggctgacctgagac (universal portion) | | |
| | | | {close oversize parenthesis} | SEQ ID NO 11 |
| | | ctctttccacctgaaagcaaggt (locus specific portion) | | |
| Pair 5 | Primer 1 | cgacgtggtggatgtgctag (universal portion) | | |
| | | | {close oversize parenthesis} | SEQ ID NO 13 |
| | | cgcagcccttgccatctg (locus specific portion) | | |
| | Primer 2 | tgacgtggctgacctgagac (universal portion) | | |
| | | | {close oversize parenthesis} | SEQ ID NO 14 |
| | | cgcagcccttgccatctc (locus specific portion) | | |
| Pair 6 | Primer 1 | cgacgtggtggatgtgctag (universal portion) | | |
| | | | {close oversize parenthesis} | SEQ ID NO 16 |
| | | cttagatgtcacctcttccatgcac (locus specific portion) | | |
| | Primer 2 | tgacgtggctgacctgagac (universal portion) | | |
| | | | {close oversize parenthesis} | SEQ ID NO 17 |
| | | cttagatgtcacctcttceatgcag (locus specific portion) | | |
| Pair 7 | Primer 1 | cgacgtggtggatgtgctag (universal portion) | | |
| | | | {close oversize parenthesis} | SEQ ID NO 19 |
| | | gcgggaggaaggaagggagg (locus specific portion) | | |
| | Primer 2 | tgacgtggctgacctgagac (universal portion) | | |
| | | | {close oversize parenthesis} | SEQ ID NO 20 |
| | | gcgggaggaaggaagggagc (locus specific portion) | | |
| Pair 8 | Primer 1 | cgacgtggtggatgtgctag (universal portion) | | |
| | | | {close oversize parenthesis} | SEQ ID NO 22 |
| | | gatgcetcttgcattgtgaacg (locus specific portion) | | |
| | Primer 2 | tgacgtggctgacctgagac (universal portion) | | |
| | | | {close oversize parenthesis} | SEQ ID NO 23 |
| | | gatgceteugcattgtgaace (locus specific portion) | | |
| Pair 9 | Primer 1 | cgacgtggtggatgtgctag (universal portion) | | |
| | | | {close oversize parenthesis} | SEQ ID NO 25 |
| | | cattgtgtttcaaacgcgtgcc (locus specific portion) | | |
| | Primer 2 | tgacgtggctgacctgagac (universal portion) | | |
| | | | {close oversize parenthesis} | SEQ ID NO 26 |
| | | cattgtgtttcaaacgcgtgct (locus specific portion) | | |
| Pair 10 | Primer 1 | cgacgtggtggatgtgctag (universal portion) | | |
| | | | {close oversize parenthesis} | SEQ ID NO 28 |
| | | tgccactctgacactgatgcttg (locus specific portion) | | |
| | Primer 2 | tgacgtggctgacctgagac (universal portion) | | |
| | | | {close oversize parenthesis} | SEQ ID NO 29 |
| | | tgccactctgacactgatgcttc (locus specific portion) | | |
| Pair 11 | Primer 1 | cgacgtggtggatgtgctag (universal portion) | | |
| | | | {close oversize parenthesis} | SEQ ID NO 31 |
| | | ctgccttggctcccagcc (locus specific portion) | | |
| | Primer 2 | tgacgtggctgacctgagac (universal portion) | | |
| | | | {close oversize parenthesis} | SEQ ID NO 32 |
| | | ctgccttggctcccagct (locus specific portion) | | |
| Pair 12 | Primer 1 | cgacgtggtggatgtgctag (universal portion) | | |
| | | | {close oversize parenthesis} | SEQ ID NO 34 |
| | | cacaaagctgatctgagcacctauac (locus specific portion) | | |
| | Primer 2 | tgacgtggctgacctgagac (universal portion) | | |
| | | | {close oversize parenthesis} | SEQ ID NO 35 |
| | | cacaaagctgatctgagcacctattag (locus specific portion) | | |
| Pair 13 | Primer 1 | cgacgtggtggatgtgctag (universal portion) | | |
| | | | {close oversize parenthesis} | SEQ ID NO 37 |
| | | gctgtgggctctatgtatcatccaac (locus specific portion) | | |
| | Primer 2 | tgacgtggctgacctgagac (universal portion) | | |
| | | | {close oversize parenthesis} | SEQ ID NO 38 |
| | | gctgtgggctctatgtatcatccaag (locus specific portion) | | |
| Pair 14 | Primer 1 | cgacgtggtggatgtgctag (universal portion) | | |
| | | | {close oversize parenthesis} | SEQ ID NO 40 |
| | | gcccaatggctaatttccttacatg (locus specific portion) | | |
| | Primer 2 | tgacgtggctgacctgagac (universal portion) | | |
| | | | {close oversize parenthesis} | SEQ ID NO 41 |
| | | gcccaatggctaatttccttacatc (locus specific portion) | | |
| Pair 15 | Primer 1 | cgacgtggtggatgtgctag (universal portion) | | |
| | | | {close oversize parenthesis} | SEQ ID NO 43 |
| | | gggaaactgctgggtctgt (locus specific portion) | | |
| | Primer 2 | tgacgtggctgacctgagac (universal portion) | | |
| | | | {close oversize parenthesis} | SEQ ID NO 44 |
| | | gggaaactgctgggtctgc (locus specific portion) | | |
| Pair 16 | Primer 1 | cgacgtggtggatgtgctag (universal portion) | | |
| | | | {close oversize parenthesis} | SEQ ID NO 46 |
| | | ctgtgcatccactgcgcc (locus specific portion) | | |
| | Primer 2 | tgacgtggctgacctgagac (universal portion) | | |
| | | | {close oversize parenthesis} | SEQ ID NO 47 |
| | | ctgtgcatccactgcgca (locus specific portion) | | |
| Pair 17 | Primer 1 | cgacgtggtggatgtgctag (universal portion) | | |
| | | | {close oversize parenthesis} | SEQ ID NO 49 |
| | | cctggagcatgagctgaccac (locus specific portion) | | |
| | Primer 2 | tgacgtggctgacctgagac (universal portion) | | |
| | | | {close oversize parenthesis} | SEQ ID NO 50 |
| | | cctggagcatgagctgaccaa (locus specific portion) | | |
| Pair 18 | Primer 1 | cgacgtggtggatgtgctag (universal portion) | | |
| | | | {close oversize parenthesis} | SEQ ID NO 52 |
| | | ccatgcctcacctcctgcatt (locus specific portion) | | |
| | Primer 2 | tgacgtggctgacctgagac (universal portion) | | |
| | | | {close oversize parenthesis} | SEQ ID NO 53 |
| | | ccatgcctcacctcctgcatc (locus specific portion) | | |
| Pair 19 | Primer 1 | cgacgtggtggatgtgctag (universal portion) | | |
| | | | {close oversize parenthesis} | SEQ ID NO 55 |
| | | gcatgccattgccaaaucc (locus specific portion) | | |
| | Primer 2 | tgacgtggctgacctgagac (universal portion) | | |
| | | | {close oversize parenthesis} | SEQ ID NO 56 |
| | | gcatgccattgccaaattct (locus specific portion) | | |
| Pair 20 | Primer 1 | cgacgtggtggatgtgctag (universal portion) | | |
| | | | {close oversize parenthesis} | SEQ ID NO 58 |
| | | gccaaccagacctcccagg (locus specific portion) | | |
| | Primer 2 | tgacgtggctgacctgagac (universal portion) | | |
| | | | {close oversize parenthesis} | SEQ ID NO 59 |
| | | gccaaccagacctcccagc (locus specific portion) | | |
| Pair 21 | Primer 1 | cgacgtggtggatgtgctag (universal portion) | | |
| | | | {close oversize parenthesis} | SEQ ID NO 61 |
| | | gggagacaggcccatgca (locus specific portion) | | |
| | Primer 2 | tgacgtggctgacctgagac (universal portion) | | |
| | | | {close oversize parenthesis} | SEQ ID NO 62 |
| | | gggagacaggcccatgct (locus specific portion) | | |
| Pair 22 | Primer 1 | cgacgtggtggatgtgctag (universal portion) | | |
| | | | {close oversize parenthesis} | SEQ ID NO 64 |
| | | cagaaaaggcaggaacctggaca (locus specific portion) | | |
| | Primer 2 | tgacgtggctgacctgagac (universal portion) | | |
| | | | {close oversize parenthesis} | SEQ ID NO 65 |
| | | cagaaaaggcaggaacctggact (locus specific portion) | | |
| Pair 23 | Primer 1 | cgacgtggtggatgtgctag (universal portion) | | |
| | | | {close oversize parenthesis} | SEQ ID NO 67 |
| | | gagccaagaatcgcagggaa (locus specific portion) | | |
| | Primer 2 | tgacgtggctgacctgagac (universal portion) | | |
| | | | {close oversize parenthesis} | SEQ ID NO 68 |
| | | gagccaagaatcgcagggat (locus specific portion) | | |
| Pair 24 | Primer 1 | cgacgtggtggatgtgctag (universal portion) | | |
| | | | {close oversize parenthesis} | SEQ ID NO 70 |
| | | tgtgcatgttccctggtgttca (locus specific portion) | | |
| | Primer 2 | tgacgtggctgacctgagac (universal portion) | | |
| | | | {close oversize parenthesis} | SEQ ID NO 71 |
| | | tgtgcatgttccctggtgttct (locus specific portion) | | |
| Pair 25 | Primer 1 | cgacgtggtggatgtgctag (universal portion) | | |
| | | | {close oversize parenthesis} | SEQ ID NO 73 |
| | | ggcatacccatgtgactaaatgttga (locus specific portion) | | |
| | Primer 2 | tgacgtggctgacctgagac (universal portion) | | |
| | | | {close oversize parenthesis} | SEQ ID NO 74 |
| | | ggcatacccatgtgactaaatgttgt (locus specific portion) | | |
| Pair 26 | Primer 1 | cgacgtggtggatgtgctag (universal portion) | | |
| | | | {close oversize parenthesis} | SEQ ID NO 76 |
| | | gcaaggcccaaagcaaagaa (locus specific portion) | | |
| | Primer 2 | tgacgtggctgacctgagac (universal portion) | | |
| | | | {close oversize parenthesis} | SEQ ID NO 77 |
| | | gcaaggcccaaagcaaagat (locus specific portion) | | |
| Pair 27 | Primer 1 | cgacgtggtggatgtgctag (universal portion) | | |
| | | | {close oversize parenthesis} | SEQ ID NO 79 |
| | | aggacagtggcttctgtactgcta (locus specific portion) | | |
| | Primer 2 | tgacgtggctgacctgagac (universal portion) | | |
| | | | {close oversize parenthesis} | SEQ ID NO 80 |
| | | aggacagtggcttctgtactgctt (locus specific portion) | | |
| Pair 28 | Primer 1 | cgacgtggtggatgtgctag (universal portion) | | |
| | | | {close oversize parenthesis} | SEQ ID NO 82 |
| | | ctggaagggctttgtttgccaa (locus specific portion) | | |
| | Primer 2 | tgacgtggctgacctgagac (universal portion) | | |
| | | | {close oversize parenthesis} | SEQ ID NO 83 |
| | | ctggaagggctttgtttgccat (locus specific portion) | | |
| Pair 29 | Primer 1 | cgacgtggtggatgtgctag (universal portion) | | |
| | | | {close oversize parenthesis} | SEQ ID NO 85 |
| | | gggggtactggggagaccaa (locus specific portion) | | |
| | Primer 2 | tgacgtggctgacctgagac (universal portion) | | |
| | | | {close oversize parenthesis} | SEQ ID NO 86 |
| | | gggggtactggggagaccat (locus specific portion) | | |

or sequences at least 75% homologous thereto in respect of the universal portion.

7. A mixture according to any preceding claims in which the mixture includes at least 5 primer pairs having or including one of the sequences of a primer pair selected from pair 1, pair 2, pair 3, pair 7, pair 8, pair 9, pair 10, pair 11, pair 12, pair 13, pair 14, pair 15, pair 16, pair 17, pair 18, pair 19, pair 20, pair 21, pair 22, pair 23, pair 24, pair 25, pair 26, pair 27, pair 28, pair 29 as provided in claim 1.

8. A mixture according to any preceding claims in which the mixture includes at least 8 primer pairs having or including one of the sequences of a primer pair selected from pair 1, pair 2, pair 3, pair 7, pair 8, pair 9, pair 10, pair 11, pair 12, pair 13, pair 14, pair 15, pair 16, pair 17, pair 18, pair 19, pair 20, pair 21, pair 22, pair 23, pair 24, pair 25, pair 26, pair 27, pair 28, pair 29 as provided in claim 1.

9. A method of investigating single nucleotide polymorphisms in a sample of DNA, the method comprising contacting the DNA containing sample with at least one set of first primers, amplifying the DNA using those primers to give an amplified product, contacting at least a portion of the amplified product with at least one second set of primers, amplifying the DNA using those second set of primers to give a further amplified product and examining one or more characteristics of the further amplified product, in which the first primers include one or more pairs of primers pairs, at least one of those primer pairs being a primer pair having or including the following sequences:

| | | | |
|---|---|---|---|
| Pair 1 | Primer 1 | cgacgtggtggatgtgctagccagatgtttctcaagtggtcctg | SEQ ID NO 1 |
| | Primer 2 | tgacgtggctgacctgagacaaagtggtttctcaagtggtccca | SEQ ID NO 2 |
| Pair 2 | Primer 1 | cgacgtggtggatgtgctagcctctgctttaaccacaccagca | SEQ ID NO 4 |
| | Primer 2 | tgacgtggctgacctgagaccctctgctttaaccacaccagcc | SEQ ID NO 5 |
| Pair 3 | Primer 1 | cgacgtggtggatgtgctagaccaaccccacaaagcagg | SEQ ID NO 7 |
| | Primer 2 | tgacgtggctgacctgagacaccaaccccacaaagcagc | SEQ ID NO 8 |
| Pair 4 | Primer 1 | cgacgtggtggatgtgctagctctttccacctgaaagcaaggg | SEQ ID NO 10 |
| | Primer 2 | tgacgtggctgacctgagacctctttccacctgaaagcaaggt | SEQ ID NO 11 |
| Pair 5 | Primer 1 | cgacgtggtggatgtgctagcgcagcccttgccatctg | SEQ ID NO 13 |
| | Primer 2 | tgacgtggctgacctgagaccgcagcccttgccatctc | SEQ ID NO 14 |
| Pair 6 | Primer 1 | cgacgtggtggatgtgctagcttagatgtcacctcttccatgcac | SEQ ID NO 16 |
| | Primer 2 | tgacgtggctgacctgagaccttagatgtcacctcttccatgcag | SEQ ID NO 17 |
| Pair 7 | Primer 1 | cgacgtggtggatgtgctaggcgggaggaaggaagggagg | SEQ ID NO 19 |
| | Primer 2 | tgacgtggctgacctgagacgcgggaggaaggaagggagc | SEQ ID NO 20 |
| Pair 8 | Primer 1 | cgacgtggtggatgtgctaggatgcctcttgcattgtgaacg | SEQ ID NO 22 |
| | Primer 2 | tgacgtggctgacctgagacgatgcctcttgcattgtgaacc | SEQ ID NO 23 |
| Pair 9 | Primer 1 | cgacgtggtggatgtgctagcattgtgtttcaaacgcgtgcc | SEQ ID NO 25 |
| | Primer 2 | tgacgtggctgacctgagaccattgtgtttcaaacgcgtgct | SEQ ID NO 26 |
| Pair 10 | Primer 1 | cgacgtggtggatgtgctagtgccactctgacactgatgcttg | SEQ ID NO 28 |
| | Primer 2 | tgacgtggctgacctgagactgccactctgacactgatgcttc | SEQ ID NO 29 |
| Pair 11 | Primer 1 | cgacgtggtggatgtgctagctgccttggctcccagcc | SEQ ID NO 31 |
| | Primer 2 | tgacgtggctgacctgagacctgccttggctcccagct | SEQ ID NO 32 |
| Pair 12 | Primer 1 | cgacgtggtggatgtgctagcacaaagctgatctgagcacctattac | SEQ ID NO 34 |
| | Primer 2 | tgacgtggctgacctgagaccacaaagctgatctgagcacctattag | SEQ ID NO 35 |
| Pair 13 | Primer 1 | cgacgtggtggatgtgctaggctgtgggctctatgtatcatccaac | SEQ ID NO 37 |
| | Primer 2 | tgacgtggctgacctgagacgctgtgggctctatgtatcatccaag | SEQ ID NO 38 |
| Pair 14 | Primer 1 | cgacgtggtggatgtgctaggcccaatggctaatttccttacatg | SEQ ID NO 40 |
| | Primer 2 | tgacgtggctgacctgagacgcccaatggctaatttccttacatc | SEQ ID NO 41 |
| Pair 15 | Primer 1 | cgacgtggtggatgtgctaggggaaactgctgggtctgt | SEQ ID NO 43 |
| | Primer 2 | tgacgtggctgacctgagacgggaaactgctgggtctgc | SEQ ID NO 44 |
| Pair 16 | Primer 1 | cgacgtggtggatgtgctagctgtgcatccactgcgcc | SEQ ID NO 46 |
| | Primer 2 | tgacgtggctgacctgagacctgtgcatccactgcgca | SEQ ID NO 47 |
| Pair 17 | Primer 1 | cgacgtggtggatgtgctagcctggagcatgagctgaccac | SEQ ID NO 49 |
| | Primer 2 | tgacgtggctgacetgagaccctggagcatgagctgaccaa | SEQ ID NO 50 |
| Pair 18 | Primer 1 | cgacgtggtggatgtgctagccatgcctcacctcctgcatt | SEQ ID NO 52 |
| | Primer 2 | tgacgtggctgacctgagacccatgcctcacctcctgcatc | SEQ ID NO 53 |
| Pair 19 | Primer 1 | cgacgtggtggatgtgctaggcatgccattgccaaattcc | SEQ ID NO 55 |
| | Primer 2 | tgacgtggctgacctgagacgcatgccattgccaaattct | SEQ ID NO 56 |
| Pair 20 | Primer 1 | cgacgtggtggatgtgctaggccaaccagacctcccagg | SEQ ID NO 58 |
| | Primer 2 | tgacgtggctgacctgagacgccaaccagacctcccagc | SEQ ID NO 59 |
| Pair 21 | Primer 1 | cgacgtggtggatgtgctaggggagacaggcccatgca | SEQ ID NO 61 |
| | Primer 2 | tgacgtggctgacctgagacgggagacaggcccatgct | SEQ ID NO 62 |
| Pair 22 | Primer 1 | cgacgtggtggatgtgctagcagaaaaggcaggaacctggaca | SEQ ID NO 64 |
| | Primer 2 | tgacgtggctgacctgagaccagaaaaggcaggaacctggact | SEQ ID NO 65 |
| Pair 23 | Primer 1 | cgacgtggtggatgtgctaggagccaagaatcgcagggaa | SEQ ID NO 67 |
| | Primer 2 | tgacgtggctgacctgagacgagccaagaatcgcagggat | SEQ ID NO 68 |
| Pair 24 | Primer 1 | cgacgtggtggatgtgctagtgtgcatgttccctggtgttca | SEQ ID NO 70 |
| | Primer 2 | tgacgtggctgacctgagactgtgcatgttccctggtgttct | SEQ ID NO 71 |
| Pair 25 | Primer 1 | cgacgtggtggatgtgctagggcatacccatgtgactaaatgttga | SEQ ID NO 73 |
| | Primer 2 | tgacgtggctgacctgagacggcatacccatgtgaetaaatgttgt | SEQ ID NO 74 |
| Pair 26 | Primer 1 | cgacgtggtggatgtgctaggcaaggcccaaagcaaagaa | SEQ ID NO 76 |
| | Primer 2 | tgacgtggctgacctgagacgcaaggcccaaagcaaagat | SEQ ID NO 77 |
| Pair 27 | Primer 1 | cgacgtggtggatgtgctagaggacagtggcttctgtactgcta | SEQ ID NO 79 |
| | Primer 2 | tgacgtggctgacctgagacaggacagtggcttctgtactgctt | SEQ ID NO 80 |
| Pair 28 | Primer 1 | cgacgtggtggatgtgctagctggaagggctttgtttgccaa | SEQ ID NO 82 |
| | Primer 2 | tgacgtggctgacctgagacctggaagggctttgtttgccat | SEQ ID NO 83 |
| Pair 29 | Primer 1 | cgacgtggtggatgtgctaggggggtactggggagaccaa | SEQ ID NO 85 |
| | Primer 2 | tgacgtggctgacctgagacgggggtactggggagaccat | SEQ ID NO 86 |

or sequences 75% homologous therewith.


SEQUENCE LISTING (87 sequences)

SEQ ID NO: 1 - DNA, 44 bp, Artificial sequence
  Primer
cgacgtggtggatgtgctagccagatgtttctcaagtggtcctg

SEQ ID NO: 2 - DNA, 44 bp, Artificial sequence
  Primer
tgacgtggctgacctgagacaaagtggtttctcaagtggtccca

SEQ ID NO: 3 - DNA, 44 bp, Artificial sequence
  Primer
ctagctggtggctgtgctagagcttaaactgggaagctgrtggt

SEQ ID NO: 4 - DNA, 43 bp, Artificial sequence
  Primer
cgacgtggtggatgtgctagcctctgctttaaccacaccagca

SEQ ID NO: 5 - DNA, 43 bp, Artificial sequence
  Primer
tgacgtggctgacctgagaccctctgctttaaccacaccagcc

SEQ ID NO: 6 - DNA, 47 bp, Artificial sequence
  Primer
ctagctggtggctgtgctagaaaggaccaaagaagatctcattgaga

SEQ ID NO: 7 - DNA, 39 bp, Artificial sequence
  Primer
cgacgtggtggatgtgctagaccaaccccacaaagcagg

SEQ ID NO: 8 - DNA, 39 bp, Artificial sequence
  Primer
tgacgtggctgacctgagacaccaaccccacaaagcagc

SEQ ID NO: 9 - DNA, 44 bp, Artificial sequence
  Primer
ctagctggtggctgtgctagattagagcagccaagtcctgacca

SEQ ID NO: 10 - DNA, 43 bp, Artificial Sequence
  Primer
cgacgtggtggatgtgctagctctttccacctgaaagcaaggg

SEQ ID NO: 11 - DNA, 43 bp, Artificial Sequence
  Primer
tgacgtggctgacctgagacctctttccacctgaaagcaaggt

SEQ ID NO: 12 - DNA, 40 bp, Artificial Sequence
  Primer
ctagctggtggctgtgctaggatcagccacccctctagcc

SEQ ID NO: 13 - DNA, 38 bp, Artificial Sequence
  Primer
cgacgtggtggatgtgctagcgcagcccttgccatctg

SEQ ID NO: 14 - DNA, 38 bp, Artificial Sequence
  Primer
tgacgtggctgacctgagaccgcagcccttgccatctc

SEQ ID NO: 15 - DNA, 39 bp, Artificial Sequence
  Primer
ctagctggtggctgtgctagggctgtgaagtgagcctgg

SEQ ID NO: 16 - DNA, 45 bp, Artificial Sequence
  Primer
cgacgtggtggatgtgctagcttagatgtcacctcttccatgcac

SEQ ID NO: 17 - DNA, 45 bp, Artificial Sequence
  Primer
tgacgtggctgacctgagaccttagatgtcacctcttccatgcag

SEQ ID NO: 18 - DNA, 41 bp, Artificial Sequence
  Primer
ctagctggtggctgtgctagtttgaaagcacccaggaatgg

SEQ ID NO: 19 - DNA, 40 bp, Artificial sequence
  Primer
cgacgtggtggatgtgctaggcgggaggaaggaagggagg

SEQ ID NO: 20 - DNA, 40 bp, Artificial sequence
  Primer
tgacgtggctgacctgagacgcgggaggaaggaagggagc

SEQ ID NO: 21 - DNA, 40 bp, Artificial sequence
  Primer
ctagctggtggctgtgctaggggcaggtcaggatggagca

SEQ ID NO: 22 - DNA, 42 bp, Artificial sequence
  Primer
cgacgtggtggatgtgctaggatgcctcttgcattgtgaacg

SEQ ID NO: 23 - DNA, 42 bp, Artificial sequence
  Primer
tgacgtggctgacctgagacgatgcctcttgcattgtgaacc

SEQ ID NO: 24 - DNA, 46 bp, Artificial sequence
  Primer
ctagctggtggctgtgctaggctcaacagcacaactctgctacagc

SEQ ID NO: 25 - DNA, 42 bp, Artificial sequence
  Primer
cgacgtggtggatgtgctagcattgtgtttcaaacgcgtgcc

SEQ ID NO: 26 - DNA, 42 bp, Artificial sequence
  Primer
tgacgtggctgacctgagaccattgtgtttcaaacgcgtgct

SEQ ID NO: 27 - DNA, 49 bp, Artificial sequence
  Primer
ctagctggtggctgtgctagatctcaccttacatgcttatttgcaaggt

SEQ ID NO: 28 - DNA, 43 bp, Artificial sequence
  Primer
cgacgtggtggatgtgctagtgccactctgacactgatgcttg

SEQ ID NO: 29 - DNA, 43 bp, Artificial sequence
  Primer
tgacgtggctgacctgagactgccactctgacactgatgcttc

SEQ ID NO: 30 - DNA, 41 bp, Artificial sequence
  Primer
ctagctggtggctgtgctagaatggggagattggcttggac

SEQ ID NO: 31 - DNA, 38 bp, Artificial sequence
  Primer
cgacgtggtggatgtgctagctgccttggctcccagcc

SEQ ID NO: 32 - DNA, 38 bp, Artificial sequence
  Primer
tgacgtggctgacctgagacctgccttggctcccagct

SEQ ID NO: 33 - DNA, 45 bp, Artificial sequence
  Primer
ctagctggtggctgtgctagcctgaacatccctgaaggtatttcg

SEQ ID NO: 34 - DNA, 47 bp, Artificial sequence
  Primer
cgacgtggtggatgtgctagcacaaagctgatctgagcacctattac

SEQ ID NO: 35 - DNA, 47 bp, Artificial sequence
  Primer
tgacgtggctgacctgagaccacaaagctgatctgagcacctattag

SEQ ID NO: 36 - DNA, 42 bp, Artificial sequence
  Primer
ctagctggtggctgtgctagtgcctttgagaactcagtgcct

SEQ ID NO: 37 - DNA, 46 bp, Artificial sequence
  Primer
cgacgtggtggatgtgctaggctgtgggctctatgtatcatccaac

SEQ ID NO: 38 - DNA, 46 bp, Artificial sequence
  Primer
tgacgtggctgacctgagacgctgtgggctctatgtatcatccaag

SEQ ID NO: 39 - DNA, 43 bp, Artificial sequence
  Primer
ctagctggtggctgtgctagatgggaatgaagggaaaccaaga

SEQ ID NO: 40 - DNA, 45 bp, Artificial sequence
  Primer
cgacgtggtggatgtgctaggcccaatggctaatttccttacatg

SEQ ID NO: 41 - DNA, 45 bp, Artificial sequence
  Primer
tgacgtggctgacctgagacgcccaatggctaatttccttacatc

SEQ ID NO: 42 - DNA, 44 bp, Artificial sequence
  Primer
ctagctggtggctgtgctaggaggactgtacagcagcgtcagtg

SEQ ID NO: 43 - DNA, 39 bp, Artificial sequence
  Primer
cgacgtggtggatgtgctaggggaaactgctgggtctgt

SEQ ID NO: 44 - DNA, 39 bp, Artificial sequence
  Primer
tgacgtggctgacctgagacgggaaactgctgggtctgc

SEQ ID NO: 45 - DNA, 40 bp, Artificial sequence
  Primer
ctagctggtggctgtgctagaatgacctgccccacaggag

SEQ ID NO: 46 - DNA, 38 bp, Artificial sequence
  Primer
cgacgtggtggatgtgctagctgtgcatccactgcgcc

SEQ ID NO: 47 - DNA, 38 bp, Artificial sequence
  Primer
tgacgtggctgacctgagacctgtgcatccactgcgca

SEQ ID NO: 48 - DNA, 39 bp, Artificial sequence
  Primer
ctagctggtggctgtgctagtctaggctggtgccagccc

SEQ ID NO: 49 - DNA, 41 bp, Artificial sequence
  Primer
cgacgtggtggatgtgctagcctggagcatgagctgaccac

SEQ ID NO: 50 - DNA, 41 bp, Artificial sequence
  Primer
tgacgtggctgacctgagaccctggagcatgagctgaccaa

SEQ ID NO: 51 - DNA, 41 bp, Artificial sequence
  Primer
ctagctggtggctgtgctagggctctgaagaacaatgggga

SEQ ID NO: 52 - DNA, 41 bp, Artificial sequence
  Primer
cgacgtggtggatgtgctagccatgcctcacctcctgcatt

SEQ ID NO: 53 - DNA, 41 bp, Artificial sequence
  Primer
tgacgtggctgacctgagacccatgcctcacctcctgcatc

SEQ ID NO: 54 - DNA, 43 bp, Artificial sequence
  Primer
ctagctggtggctgtgctagcaatcctgtttgcagagttccag

SEQ ID NO: 55 - DNA, 40 bp, Artificial sequence
  Primer
cgacgtggtggatgtgctaggcatgccattgccaaattcc

SEQ ID NO: 56 - DNA, 40 bp, Artificial sequence
  Primer
tgacgtggctgacctgagacgcatgccattgccaaattct

SEQ ID NO: 57 - DNA, 38 bp, Artificial sequence
  Primer
ctagctggtggctgtgctagtgagccaaggtgtgggga

SEQ ID NO: 58 - DNA, 39 bp, Artificial sequence
  Primer
cgacgtggtggatgtgctaggccaaccagacctcccagg

SEQ ID NO: 59 - DNA, 39 bp, Artificial sequence
  Primer
tgacgtggctgacctgagacgccaaccagacctcccagc

SEQ ID NO: 60 - DNA, 43 bp, Artificial sequence
  Primer
ctagctggtggctgtgctagaaacacaggtctccagcttgagc

SEQ ID NO: 61 - DNA, 38 bp, Artificial sequence
  Primer
cgacgtggtggatgtgctaggggagacaggcccatgca

SEQ ID NO: 62 - DNA, 38 bp, Artificial sequence
  Primer
tgacgtggctgacctgagacgggagacaggcccatgct

SEQ ID NO: 63 - DNA, 46 bp, Artificial sequence
  Primer
ctagctggtggctgtgctaggccattcagaactaactagtctggga

SEQ ID NO: 64 - DNA, 43 bp, Artificial sequence
  Primer
cgacgtggtggatgtgctagcagaaaaggcaggaacctggaca

SEQ ID NO: 65 - DNA, 43 bp, Artificial sequence
  Primer
tgacgtggctgacctgagaccagaaaaggcaggaacctggact

SEQ ID NO: 66 - DNA, 41 bp, Artificial sequence
  Primer
ctagctggtggctgtgctagcgacgggggttgagtggttca

SEQ ID NO: 67 - DNA, 40 bp, Artificial sequence
  Primer
cgacgtggtggatgtgctaggagccaagaatcgcagggaa

SEQ ID NO: 68 - DNA, 40 bp, Artificial sequence
  Primer
tgacgtggctgacctgagacgagccaagaatcgcagggat

SEQ ID NO: 69 - DNA, 42 bp, Artificial sequence
  Primer
ctagctggtggctgtgctaggctaaagcagctctgaaaccca

SEQ ID NO: 70 - DNA, 42 bp, Artificial sequence
  Primer
cgacgtggtggatgtgctagtgtgcatgttccctggtgttca

SEQ ID NO: 71 - DNA, 42 bp, Artificial sequence
  Primer
tgacgtggctgacctgagactgtgcatgttccctggtgttct

SEQ ID NO: 72 - DNA, 39 bp, Artificial sequence
  Primer
ctagctggtggctgtgctagaacggccttgcttcgctga

SEQ ID NO: 73 - DNA, 46 bp, Artificial sequence
  Primer
cgacgtggtggatgtgctagggcatacccatgtgactaaatgttga

SEQ ID NO: 74 - DNA, 46 bp, Artificial sequence
  Primer
tgacgtggctgacctgagacggcatacccatgtgactaaatgttgt

SEQ ID NO: 75 - DNA, 45 bp, Artificial sequence
  Primer
ctagctggtggctgtgctagatggctgttagtgtgcctatcatca

SEQ ID NO: 76 - DNA, 40 bp, Artificial sequence
  Primer
cgacgtggtggatgtgctaggcaaggcccaaagcaaagaa

SEQ ID NO: 77 - DNA, 40 bp, Artificial sequence
  Primer
tgacgtggctgacctgagacgcaaggcccaaagcaaagat

SEQ ID NO: 78 - DNA, 45 bp, Artificial sequence
  Primer
ctagctggtggctgtgctagtggatagatgatcagtctgcgttct

SEQ ID NO: 79 - DNA, 44 bp, Artificial sequence
  Primer
cgacgtggtggatgtgctagaggacagtggcttctgtactgcta

SEQ ID NO: 80 - DNA, 44 bp, Artificial sequence
  Primer
tgacgtggctgacctgagacaggacagtggcttctgtactgctt

SEQ ID NO: 81 - DNA, 42 bp, Artificial sequence
  Primer
ctagctggtggctgtgctagggccagcagagattcacactgt

SEQ ID NO: 82 - DNA, 42 bp, Artificial sequence
  Primer
cgacgtggtggatgtgctagctggaagggctttgtttgccaa

SEQ ID NO: 83 - DNA, 42 bp, Artificial sequence
  Primer
tgacgtggctgacctgagacctggaagggctttgtttgccat

SEQ ID NO: 84 - DNA, 43 bp, Artificial sequence
  Primer
ctagctggtggctgtgctaggatggaatcactgtccttgccct

SEQ ID NO: 85 - DNA, 40 bp, Artificial sequence
  Primer
cgacgtggtggatgtgctaggggggtactggggagaccaa

SEQ ID NO: 86 - DNA, 40 bp, Artificial sequence
  Primer
tgacgtggctgacctgagacgggggtactggggagaccat

SEQ ID NO: 87 - DNA, 41 bp, Artificial sequence
  Primer
ctagctggtggctgtgctagcggaggagattttgccctgca